(19) United States
(12) Patent Application Publication
(10) Pub. No.: US 2024/0285776 A1
(43) Pub. Date: Aug. 29, 2024

(54) SMALL MOLECULE-BASED BI-SPECIFIC IMMUNE CELL TETHERS AND THEIR USE IN THE TREATMENT OF ENVELOPED VIRUS INFECTION

(71) Applicant: PURDUE RESEARCH FOUNDATION, West Lafayette, IN (US)

(72) Inventors: Philip LOW, West Lafayette, IN (US); Imrul SHAHRIAR, West Lafayette, IN (US)

(21) Appl. No.: 18/560,670

(22) PCT Filed: May 13, 2022
(86) PCT No.: PCT/US2022/029267

§ 371 Date: Nov. 13, 2023

Related U.S. Application Data
(60) Provisional application No. 63/188,892, filed on May 14, 2021.

(51) Int. Cl. A61K 47/55; A61P 31/16

(52) U.S. Cl. CPC A61K 47/55; A61P 31/16

(57) ABSTRACT

A conjugate of formula EVTsmL-L-AMRL, wherein EVTsmL is an enveloped virus-targeting, small molecule ligand, AMRL is an activated macrophage-recruiting ligand, and L is a linker that is covalently bound to EVTsmL and AMRL; a composition comprising the same; and a method of using the conjugate or composition to treat (therapeutically or prophylactically) a viral infection.

### PRIORITY

This patent application is related to and claims the priority benefit of U.S. Provisional Patent Application No. 63/188,892 filed May 14, 2021, the content of which is hereby incorporated by reference in its entirety into this disclosure.

### TECHNICAL FIELD

This disclosure relates to enveloped viruses, small molecule antiviral agents, immune cells (e.g., activated macrophages), and prophylactic and therapeutic treatment of infection with an enveloped virus.

### BACKGROUND

An enveloped virus is a virus that has an envelope derived from the plasma membrane of a cell infected by the virus. The plasma membrane envelopes newly formed virions as they emerge from the infected cell. The envelope may play a role in helping a virus survive and infect other cells.

The International Committee on Taxonomy of Viruses (ICTV) has developed a single system of classification and nomenclature that covers all viruses. Developed by virologists, the system classifies viruses by order (-virales)→family (-viridae)→subfamily (-virinae)→genus (-virus)→species, which further includes subspecies, strains, and variants, which are used for diagnosis and vaccine development.

Enveloped viruses cause disease in vertebrates. Of the more than 4,000 different viruses with 30,000 strains/subtypes, several hundred cause disease in humans.

Viruses are typically classified by phenotypic characteristics, such as morphology, nucleic acid type, mode of replication, host organisms, and the type of disease(s) they cause. Informally, viruses can be classified in seven different categories: enteric viruses, respiratory viruses, arboviruses, blood-borne viruses, sexually transmitted viruses, hepatitis viruses, and oncogenic viruses.

Enteric viruses are acquired by ingestion (fecal-oral transmission) and replicate primarily in the intestinal tract. Respiratory viruses are acquired by inhalation (e.g., respiratory transmission) or by fomites (e.g., inanimate objects carrying virus) and primarily replicate in the respiratory tract. Arboviruses replicate in hematophagous (e.g., blood-feeding) arthropod hosts, such as mosquitoes and ticks, and are then transmitted by bite to vertebrates, where the virus replicates and produces viremia of sufficient magnitude to infect other blood-feeding arthropods. Blood-borne viruses are transmitted by transfusion of blood or blood products, by sharing of intravenous injecting equipment, and parenteral transfer of blood or body fluids, whereas sexually transmitted viruses are transmitted by sexual contact.

While hepatitis viruses all affect the liver, this category includes a variety of viruses (e.g., Hepatitis A, B, C, D, and E viruses) that belong to completely unrelated taxonomic families. The main target of hepatitis viruses is liver cells because molecules on the surface of liver cells match corresponding molecules on the surfaces of such viruses. Oncogenic viruses can cause persistent infection and may transform host cells, which, in turn, may become malignant.

An alternative classification system is based on the viral genome. See, for example, Tables 1 and 2.

TABLE 1
DNA Viruses

| | |
|---|---|
| dsDNA | Adenoviridae |
| | Asfarviridae |
| | Alloherpesviridae |
| | Herpesviridae |
| | Iridoviridae |
| | Papillomaviridae (e.g., human papilloma virus (HPV)) |
| | Polyomaviridae |
| | Poxviridae |
| ssDNA | Anelloviridae |
| | Circoviridae |
| | Parvoviridae |
| dsDNA (RT) | Hepadnaviridae (e.g., hepatitis B virus (HBV)) |

TABLE 2
RNA Viruses

| | |
|---|---|
| dsRNA | Birnaviridae |
| | Picobirnaviridae |
| | Reoviradae (Sedoreovirinae; Spinareovirinae) |
| ssRNA | Arenaviridae |
| (−) and | Deltavirus |
| (+/−) | Bunyaviridae (includes Rift Valley fever virus) |
| | Othomyxoviridae (influenza) |
| | Mononegavirales (Filoviridae, Bornoviridae, Paramyxoviridae |
| | (respiratory syncytial virus (RSV)), and Rhabdoviridae) |
| ssRNA | Astroviridae |
| (+) | Caliciviridae |
| | Flaviviridae (e.g., hepatitis C virus (HCV)) |
| | Hepeviridae |
| | Nodaviridae |
| | Picornaviridae (e.g., hepatitis A virus (HAV)) |
| | Togaviridae |
| | Nidovirales (Arteriviridae and Coronaviridae (coronavirus) |
| | (Coronavirinae and Torovirinae)) |
| ssRNA | Metaviridae |
| (RT) | Retroviridae (e.g., human immunodeficiency virus (HIV)) |

Currently, viral infection can be treated by vaccines, directly acting antiviral small molecules, monoclonal antibodies, immunomodulators, and bispecific T-cell-engaging (BITE) antibody conjugates. While all these methods can be effective, they also present disadvantages. For example, vaccines may not provide protection against rapidly mutating viruses. Directly acting, antiviral, small molecules are not capable of engaging the immune system. Antibodies can be expensive, involve complex manufacturing, and, due to their large molecular weight, may not be able to penetrate target tissues as well as an active agent with a smaller molecular weight.

In view of the above, a conjugate, as well as compositions and methods of use, for the prophylactic and therapeutic treatment of infection with an enveloped virus is needed. This and other objects, as well as advantages and inventive features, will be apparent from the detailed description provided herein.

### SUMMARY

Provided is a conjugate of formula I.

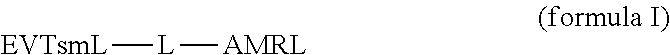

wherein EVTsmL is a radical of an enveloped virus-targeting, small molecule ligand, AMRL is a radical of an activated macrophage-recruiting ligand, and L is a linker that is covalently bound to EVTsmL and AMRL. In certain embodiments, the AMRL can bind folate receptor 3 (FRβ) on an activated macrophage.

The EVTsmL (e.g., zanamivir, oseltamivir, peramivir or laninamivir) can bind neuraminidase (NA) or hemagglutinin (HA) on an influenza virus or an influenza virus-infected cell. In certain embodiments, the EVTsmL is zanamivir. In certain embodiments, the EVTsmL is oseltamivir, peramivir or laninamivir.

The EVTsmL can bind human gp120 on human immunodeficiency virus (HIV) or an HIV-infected cell. The EVTsmL can bind CD38 on a latently virus-infected cell. The EVTsmL can bind hepatitis B surface antigen (HBsAg) or hepatitis B core antigen (HBcAg) on hepatitis B virus (HBV) or an HBV-infected cell. The EVTsmL can bind fusion protein F on respiratory syncytial virus (RSV) or an RSV-infected cell. The EVTsmL can bind spike protein on coronavirus or a coronavirus-infected cell.

L can comprise a spacer and a cleavable bridge between EVTsmL and AMRL. Alternatively, L can comprise a spacer and a non-cleavable bridge between EVTsmL and AMRL. L can make the conjugate more water-soluble. L can comprise one or more of an amino acid, a polyethylene glycol (PEG) monomer, a PEG oligomer, a PEG polymer, or a combination of any of the foregoing. L can comprise an oligomer of peptidoglycans, glycans, anions, or a combination of any of the foregoing. L can comprise at least one 2,3-diaminopropionic acid, at least one glutamic acid, and/or at least one cysteine. L can comprise an oligomer of peptidoglycans, glycans, an analog of folic acid or an antifolate.

The AMRL can be a folate radical, a folate derivative radical, a folate analog radical, or an antifolate radical. The AMRL can be (or be a radical of) folic acid, a derivative of folic acid, an analog of folic acid, or an antifolate. The AMRL can comprise a pteroyl amino acid (or a radical thereof). The AMRL can comprise a pteroyl amino acid radical, such as pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine (or a radical of any of the foregoing).

In certain embodiments, L comprises one or more of an amino acid, a PEG monomer, a PEG oligomer, a PEG polymer, or a combination of any of the foregoing; and AMRL is folic acid, a derivative of folic acid, an analog of folic acid, or an antifolate (or a radical of any of the foregoing). In certain embodiments, L comprises an oligomer of peptidoglycans, glycans, anions, or a combination thereof, and AMRL is folic acid, a derivative of folic acid, an analogue of folic acid, or an antifolate (or a radical of any of the foregoing). In certain embodiments, L comprises one or more of an amino acid, a PEG monomer, a PEG oligomer, a PEG polymer, or a combination of any of the foregoing; and AMRL comprises a pteroyl amino acid (e.g., AMRL is a pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine). In certain embodiments, L comprises an oligomer of peptidoglycans, glycans, anions, or a combination thereof, and AMRL comprises a pteroyl amino acid. In certain embodiments, L comprises PEG₃, PEG₆, or PEG₁₁. In certain embodiments, L can further comprise a rigid moiety, such as dibenzocyclooctyne (DBCO). Thus, in some embodiments, L comprises PEGₙ-DBCO-PEGₙ, wherein each n independently is an integer between 1-15, such as PEG₃-DBCO-PEG₃, PEG₆-DBCO-PEG₆, or PEG₁₁-DBCO-PEG₁₁.

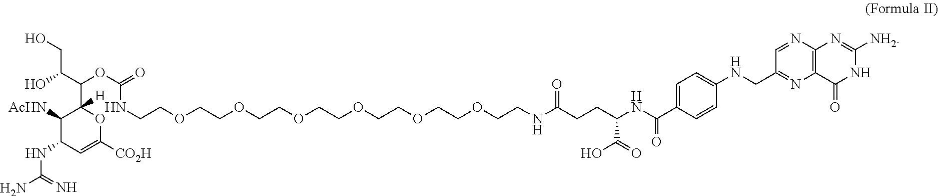

The conjugate can have the structure of formula II:

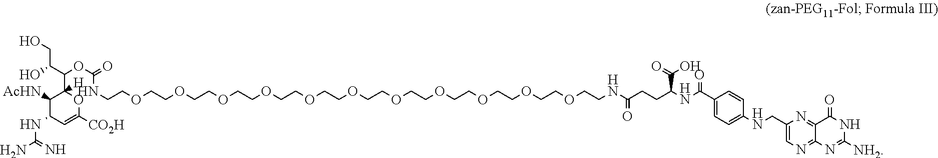

The conjugate can have the structure of formula IV:

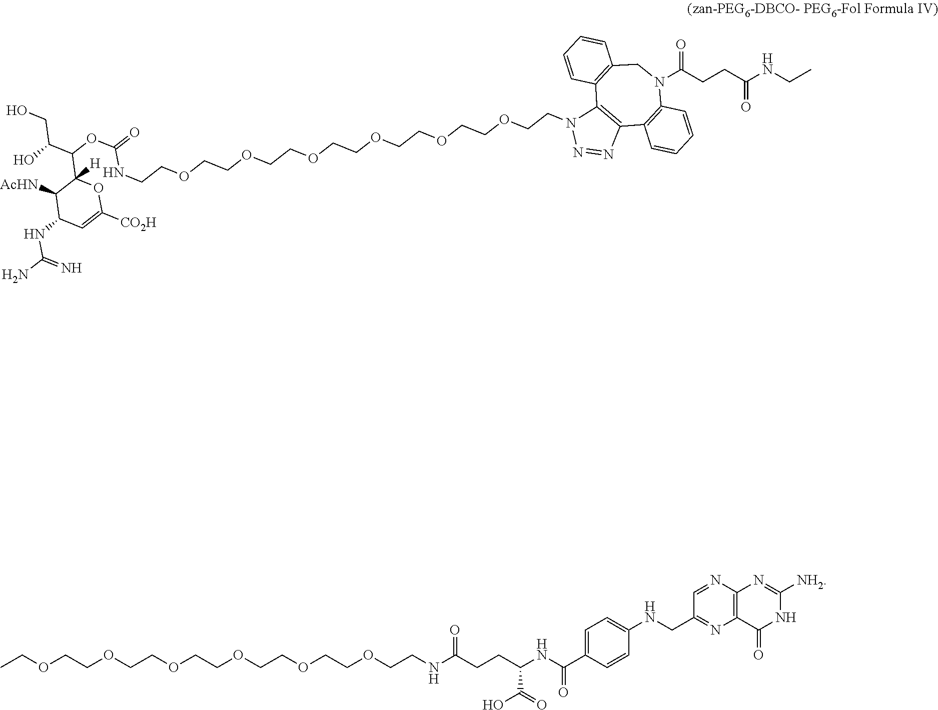

Further provided is a composition comprising an antivirally effective amount of a conjugate. In certain embodiments, the composition can further comprise a pharmaceutically acceptable excipient. Additionally or alternatively, the composition can comprise one or more active agents.

Still further provided is a method of treating prophylactically or therapeutically a viral infection in a subject. The method comprises administering to the subject an antivirally effective amount of (i) a conjugate of formula I.

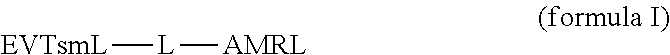

wherein EVTsmL is a radical of an enveloped virus-targeting, small molecule ligand, AMRL is a radical of an activated macrophage-recruiting ligand, and L is a linker that is covalently bound to EVTsmL and AMRL, or (ii) a composition comprising an antivirally effective amount of the conjugate. In certain embodiments of the method, AMRL of the conjugate can bind FRβ on an activated macrophage. In certain embodiments of the method, EVTsmL binds NA or HA on an influenza virus or an influenza virus-infected cell (e.g., EVTsmlL is zanamivir). In certain embodiments, EVTsmL is zanamivir, oseltamivir, peramivir, or laninamivir.

In certain embodiments of the method of treating (e.g., prophylactically or therapeutically) a viral infection in a subject, EVTsmL of the conjugate binds human gp120 on HIV or an HIV-infected cell. In certain embodiments, EVTsmL of the conjugate binds CD38 on a latently virus-infected cell. In certain embodiments, EVTsmL of the conjugate binds HBsAG or HBcAg on an HBV or HBV-infected cell. In certain embodiments, EVTsmL binds fusion protein F on an RSV or RSV-infected cell. In certain embodiments, EVTsmL of the conjugate binds spike protein on a coronavirus or a coronavirus-infected cell.

L of the conjugate administered can comprise a spacer and a cleavable bridge between EVTsmL and AMRL. Alternatively, L of the conjugate can comprise a spacer and a non-cleavable bridge between EVTsmL and AMRL. The structure of L can be selected to enhance the water-solubility of the conjugate. In certain embodiments of the method, L of the conjugate comprises one or more of an amino acid, a PEG monomer, a PEG oligomer, a PEG polymer, or a combination of any of the foregoing.

In certain embodiments, L comprises an oligomer of peptidoglycans, glycans, anions, or a combination thereof. In certain embodiments of the method, AMRL can be folic acid, a derivative of folic acid, an analog of folic acid, or an antifolate (or a radical of any of the foregoing).

In certain embodiments, L comprises one or more of an amino acid, a PEG monomer, a PEG oligomer, a PEG polymer, or a combination of any of the foregoing, and AMRL is folic acid, a derivative of folic acid, an analog of folic acid, or an antifolate (or a radical of any of the foregoing. In certain embodiments of the method, AMRL of the conjugate comprises a pteroyl amino acid (e.g., pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine).

In certain embodiments, L comprises PEG₃, PEG₆, or PEG₁₁. In certain embodiments, L can further comprise a rigid moiety, such as DBCO. Thus, in some embodiments, L comprises PEGₙ-DBCO-PEGₙ, wherein each n independently is an integer between 1-15, such as PEG₃-DBCO-PEG₃, PEG₆-DBCO-PEG₆, or PEG₁₁-DBCO-PEG₁₁.

The method of treating prophylactically or therapeutically a viral infection in a subject can comprise administering to the subject an antivirally effective amount of (i) a conjugate of formula II:

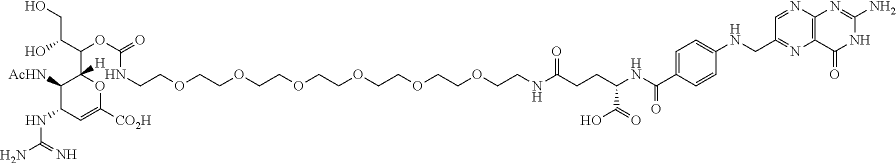

(Formula II), or (ii) a composition comprising an antivirally effective amount of the conjugate.

The method of treating prophylactically or therapeutically a viral infection in a subject can comprise administering to the subject an antivirally effective amount of (i) a conjugate of formula III:

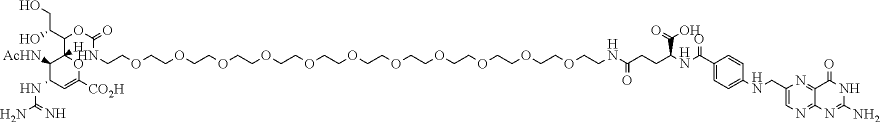

(Formula III), or (ii) a composition comprising an antivirally effective amount of the conjugate.

The method of treating prophylactically or therapeutically a viral infection in a subject can comprise administering to the subject an antivirally effective amount of (i) a conjugate of formula IV:

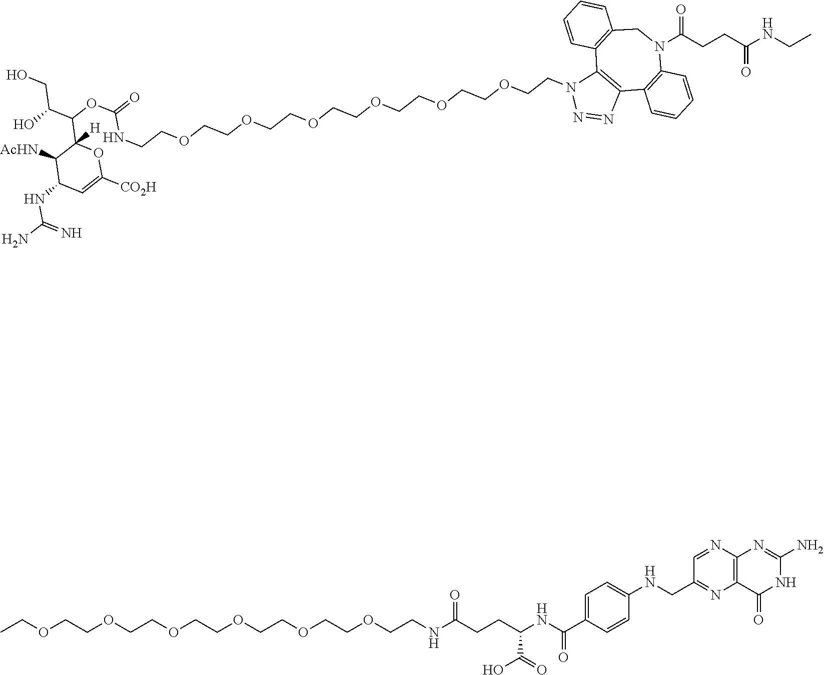

(Formula IV), or (ii) a composition comprising an antivirally effective amount of the conjugate.

### DESCRIPTION OF FIGURES

FIG. 1 shows data related to the therapeutic efficacy of zanamivir-folate in protecting mice from lethal influenza virus infections, with data labelled “treated” from the cohort that received a zanamivir-folate conjugate and the data labelled “untreated” from the control cohort.

FIG. 2 shows representative photomicrographs from the Control, ARDS and HINT groups showing CD68+ macrophages in lung parenchyma (i, j, k), and small airways (m, n, o), with the arrows indicating positive staining. The graphs on the bottom showing the density of CD68+ macrophages in the Control, ARDS and HINT groups (one-way ANOVA and Kruskal Wallis test) (Buttignol et al., Respiratory Research 18: 147 (2017); Cancer Immunol Immunother. 58: 1577-1586 (2009); and Arthritis Rheum. 52: 2666-2675 (2005)).

FIG. 3 shows the therapeutic efficacy of zanamivir-folate (zan-folate) in protecting mice from lethal influenza virus infections in a dose escalation study.

FIG. 4 shows the therapeutic efficacy of zanamvir-folate (zan-folate) in protecting mice from lethal influenza virus infections in a linker variation study.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this disclosure.

### Conjugates/Compounds

The present disclosure is directed to conjugates/compounds and compositions that tether an immune cell to a small molecule (e.g., a mini-BIT™) and their use in the prophylactic and therapeutic treatment of a subject for a viral infection (e.g., related to an enveloped virus). “Enveloped virus” means a virus in which the virus core is surrounded by an outer wrapping or envelope. The envelope can be derived from the plasma membrane of a cell infected by the virus (i.e., the host) in a process called “budding off.” During the budding process, newly formed virus particles become “enveloped” or wrapped in an outer coat that is made from a small piece of the host cell's plasma membrane. Examples of enveloped viruses include, without limitation, hepatitis B virus (HBV), influenza, RSV, coronavirus, and HIV.

In certain embodiments, the conjugates hereof comprise a small molecule ligand (or a radical thereof) conjugated to an activated macrophage-recruiting ligand (or a radical thereof) via a linker. When administered, the conjugate can engage an immune cell of the subject, e.g., an activated macrophage, to target a virus or a virally infected cell. In certain embodiments, the small molecule ligand can target a cell-surface receptor present on an enveloped virus or a cell infected with an enveloped virus. The small molecule is advantageous in this context because its small size enables the small molecule to penetrate tissues and target a virally infected cell. It also allows for control of the level of immune response provoked.

Folate receptor β (FRβ) can be expressed on activated macrophages (e.g., present at sites of inflammation), but is not typically expressed in normal tissue. Activated macrophages can be crucial in maintaining levels of pro-inflammatory signals in a variety of diseases. Thus, in certain embodiments, the activated macrophage-recruiting ligand (or radical thereof) of the conjugates disclosed herein is a ligand of FRβ—namely a folate ligand or a derivative or analog thereof. In certain embodiments, the FRβ ligand is conjugated to the small molecule ligand that binds to a cell-surface receptor present on an enveloped virus or a cell infected with an enveloped virus.

By tethering a ligand of FRβ with a small molecule or a radical thereof, the folate ligand can bind to FRβ on an immune cell of a subject (e.g., a macrophage, in particular an activated macrophage) and the small molecule, such as an antiviral small molecule, can bind to a viral antigen on a virus or a virus-infected cell. Depending on the small molecule used, a viral antigen (e.g., on a virus or a virus-infected cell), such as influenza neuraminidase (NA), influenza hemagglutinin (HA), human immunodeficiency virus (HIV) gp120, CD38 on a latently infected cell, hepatitis B surface antigen (HBsAg), hepatitis B core antigen (HBcAg), respiratory syncytial virus (RSV) fusion protein F, or spike protein on coronavirus, can be bound.

In view of the above, provided is a conjugate of formula I:

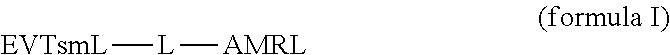

wherein EVTsmL is a radical of an enveloped virus-targeting, small molecule ligand, AMRL is a radical of an activated macrophage-recruiting ligand, and L is a linker that is covalently bound to EVTsmL and AMRL. In certain embodiments, the AMRL can bind folate receptor 3 (FRβ) on an activated macrophage, for example.

“Radical” means a fragment of a molecule, wherein that fragment has an open valence for bond formation. A monovalent radical has one open valence, such that it can form one bond with another chemical group. In some embodiments, a radical of a molecule is created by removal of one hydrogen atom from that molecule to create a monovalent radical with one open valence at the location where the hydrogen atom was removed. Where appropriate, a radical can be divalent, trivalent, etc., wherein two, three or more hydrogen atoms have been removed to create a radical which can bond to two, three, or more chemical groups. Where appropriate, a radical open valence may be created by removal of other than a hydrogen atom (e.g., a halogen atom), or by removal of two or more atoms (e.g., a hydroxyl group), as long as the atoms removed are a small fraction (20% or less of the atom count) of the total atoms in the molecule forming the radical. In some embodiments, a radical is formed from a folate, a folate derivative, a folate analog, or an antifolate by removal of a hydroxyl group.

The EVTsmL can be any small molecule that can bind to an enveloped virus or an enveloped virus antigen (e.g., a protein) displayed on the surface of a cell infected with the enveloped virus. Examples of small molecules include, but are not limited to, laninamivir (a neuraminidase inhibitor active against influenza), peramivir (a cyclopentane derivative, neuraminidase inhibitor active against influenza sold under the name Rapivab), zanamivir (a neuraminidase inhibitor active against influenza sold under the name Relenza), oseltamivir (a neuraminidase inhibitor active against influenza sold under the name Tamiflu), griffithsin (GRFT; a 121-amino acid protein isolated from the red algae Giffithsia with a Jacalin-like lectin fold that tightly binds to gp120 of HIV), enfuvirtide (a linear 36-L-amino acid synthetic peptide with an acetylated N-terminus and carboxamide C-terminus that binds to the first heptad-repeat (HR1) in the gp41 subunit of HIV and prevents fusion of viral and cellular membranes; sold under the name Fuzeon), and a 2-aminothiazolone (an inhibitor that interacts with gp120 of HIV-1 (i.e., Phe43 cavity) and inhibits interaction of gp120 with CD4 (Tiberi et al., Antimicro Agents Chemother DOI: 10.1128/AAC.02739-13 (2014)). Other examples include 5-methyl-tetrahydrofolate (5-methyl-THF), dihydrofolate reductase (DHFR) inhibitors (e.g., methotrexate (MTX) and CH-1504), thymidylate synthase (TS) inhibitors (e.g., BGC-945 and ALIMTA/pemetrexed), glycinamide ribonucleotide formyltransferase (GARTFase) inhibitors (e.g., LY309887), and Divers compounds (i.e., 6-substituted pyrrolo[2,3-d]pyrimidine thienoyl antifolates with modified amino acids (Golani et al., J Med Chem 57(19): 8152-8166 (Oct. 9 2014) and J Med Chem 59(8): 4032 (Apr. 28 2016)).

In certain embodiments, the EVTsmL, such as zanamivir, oseltamivir, peramivir or laninamivir, can bind NA or HA on an influenza virus or an influenza virus-infected cell. In certain embodiments, the EVTsmL can bind human gp120 on HIV or an HIV-infected cell. In certain embodiments, the EVTsmL can bind CD38 on a latently virus-infected cell. In certain embodiments, the EVTsmL can bind HBsAg or hepatitis B core antigen HBcAg on HBV or an HBV-infected cell. In certain embodiments, the EVTsmL can bind fusion protein F on RSV or an RSV-infected cell. In certain embodiments, the EVTsmL can bind a spike protein on coronavirus or a coronavirus-infected cell.

“Linker,” represented by “L,” generally refers to a portion of the conjugate that forms a chemical bond with the EVTsmL (e.g., the radical of an enveloped virus-targeting, small molecule ligand) and the AMRL (e.g., a radical of an activated macrophage-recruiting ligand). Illustratively, the linker can comprise atoms selected from C, N, O, S, Si, and P; C, N, O, S, and P; or C, N, O, and S. In particular, a “linker” can link two or more functional parts of a molecule to form a conjugate. The linker can have a backbone that ranges in length, such that there can be as few as two atoms in the backbone of the linker to as many as 100 or more contiguous atoms in the backbone of the linker.

The linker can be any suitable linker. For example, the linker can be a hydrophilic linker, such as a linker that comprises one or more of an amino acid (which can be the same or different), an alkyl chain, a polyethylene glycol (PEG) monomer, a PEG oligomer, a PEG polymer, or a combination of an any of the foregoing. The linker can comprise an oligomer of peptidoglycans, glycans, or anions. In some embodiments, when the linker comprises a chemical group, that group includes one or more of its atoms in the backbone of the linker. In some embodiments, the linker comprises a rigid moiety (e.g., a dibenzocyclooctyne (DBCO) moiety). In some embodiments, the linker comprises one or more PEG units and a rigid moiety. In some embodiments, the linker comprises one or more PEG units and a DBCO moiety. In certain embodiments, L is PEG₃-DBCO. In certain embodiments, L is PEG₆-DBCO. In certain embodiments, L is PEG₁₁-DBCO. In some embodiments, the linker comprises at least two sets of PEG units and a DBCO moiety (e.g., PEGₙ-DBCO-PEGₙ, wherein each n independently is an integer between 1-15). For example, and without limitation, L can be PEG₃-DBCO-PEG₃, PEG₆-DBCO-PEG₆, or PEG₁₁-DBCO-PEG₁₁.

In some embodiments, the chemical group is not required to include atoms in the backbone of L, such as when the group is for binding purposes (such as an albumin binding group), is a glucuronide, or is a “W” group as described herein. For a linker that comprises one or more PEG units, all carbon and oxygen atoms of the PEG units are part of the backbone, unless otherwise specified.

A cleavable bond for a releasable linker is part of the backbone. The “backbone” of the linker L is the shortest chain of contiguous atoms forming a covalently bonded connection between EVTsmL and AMRL of the conjugate. In some embodiments, a polyvalent linker has a branched backbone, with each branch serving as a section of backbone linker until reaching a terminus.

L can have any suitable length and chemical composition. For example, L can have a chain length of at least about 7 atoms in length. In some embodiments, L is at least about 10 atoms in length. In some embodiments, L is at least about 14 atoms in length. In some embodiments, L is between about 7 and about 31 (such as, about 7 to 31, 7 to about 31, or 7 to 31) between about 7 and about 24 (such as, about 7 to 24, 7 to about 24, or 7 to 24), or between about 7 and about 20 (such as, about 7 to 20, 7 to about 20, or 7 to 20) atoms in length. In some embodiments, L is between about 14 and about 31 (such as, about 14 to 31, 14 to about 31, or 14 to 31), between about 14 and about 24 (such as, about 14 to 24, 14 to about 24, or 14 to 24), or between about 14 and about 20 (such as, about 14 to 20, 14 to about 20, or 14 to 20) atoms in length. In some embodiments, L has a chain length of at least 7 atoms, at least 14 atoms, at least 20 atoms, at least 25 atoms, at least 30 atoms, at least 40 atoms; or from 1 to 15 atoms, 1 to 5 atoms, 5 to 10 atoms, 5 to 20 atoms, 10 to 40 atoms, or 25 to 100 atoms. An example of an L linker group having a chain length of 1 to 5 atoms is a group of the formula:

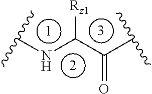

wherein R_{z1} is H, alkyl, arylalkyl, or -alkyl-S-alkyl or the side chain of any naturally or non-naturally occurring amino acid, and the like; and the numbers represent the atoms that are counted as being part of the chain, which in this case is 3 atoms. Examples of R_{z1} include H (i.e., side chain of glycine), alkyl (e.g., side chain of alanine, valine, isoleucine, and leucine), -alkyl-S-alkyl (e.g., side chain of methionine), arylalkyl (e.g., side chain of phenylalanine, tyrosine, and tryptophan), and the like. In some embodiments, the atom to which R_{z1} is attached is chiral and can have any suitable relative configuration, such as a D- or L-configuration.

The atoms used in forming L can be combined in all chemically relevant ways, such as chains of carbon atoms forming alkylene groups, chains of carbon and oxygen atoms forming polyoxyalkylene groups, chains of carbon and nitrogen atoms forming polyamines, and others. In addition, it is to be understood that the bonds connecting atoms in the chain can be either saturated or unsaturated, such that, for example, alkanes, alkenes, alkynes, cycloalkanes, arylenes, imides, and the like can be, for example, divalent radicals that are included in L. In addition, the atoms forming the linker can be cyclized upon each other to form saturated or unsaturated divalent cyclic radicals in the linker, such as radicals of the formulae:

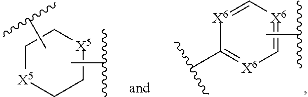

wherein each X⁵ is independently CH₂, N (when there is a bond attached to X⁵), NH or O and each X⁶ is independently N, C (when there is a bond attached to X⁶) or CH. Thus, for example, the foregoing groups can be of the formulae:

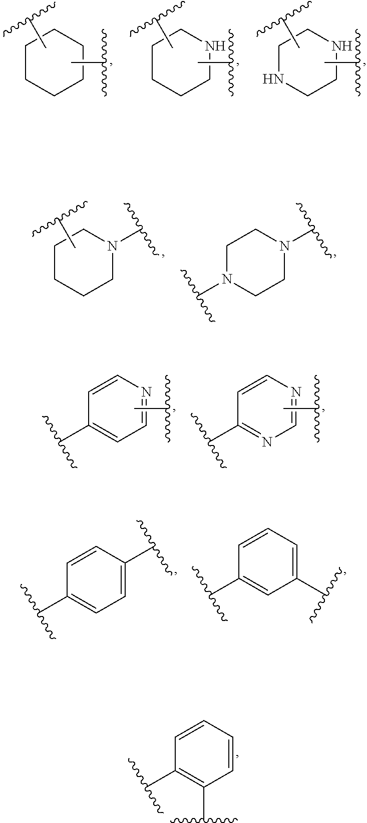

or the like. In each of the foregoing and other L groups described herein, the chain forming the linker can be substituted or unsubstituted.

Alternatively, or in addition to chain length, in some embodiments L has suitable substituents that can affect the hydrophobicity or hydrophilicity of L. Thus, for example, L can have a hydrophobic side chain group, such as an alkyl, cycloalkyl, aryl, arylalkyl, or like group, each of which is optionally substituted. If L were to include one or more amino acids, L can contain hydrophobic amino acid side chains, such as one or more amino acid side chains from phenylalanine (Phe) and tyrosine (Tyr), including substituted variants thereof, and analogs and derivatives of such side chains. Variants, analogs, and derivatives of these side chains include, for example, groups such as:

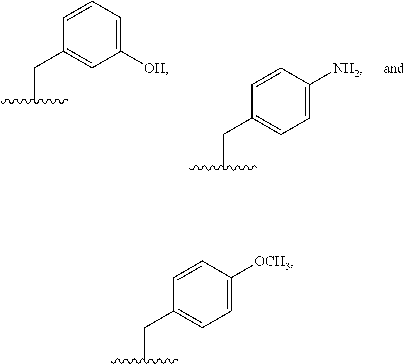 [SMILES: *Cc1cccc(O)c1 ; *Cc1ccc(N)cc1 ; *Cc1ccc(OC)cc1]

which are respectively a variant of tyrosine, an amine analog of tyrosine, and a methoxy derivative of tyrosine. Other variants, analogs, and derivatives are contemplated.

In some embodiments, L comprises one or more portions that are neutral under physiological conditions. In some embodiments, L comprises one or more portions that can be protonated or deprotonated to carry one or more positive or one or more negative charges, respectively. In some embodiments, L comprises neutral portions and portions that can be protonated to carry one or more positive charges. Examples of neutral portions include polyhydroxyl groups, such as sugars, carbohydrates, saccharides, inositols, and the like, and/or polyether groups, such as polyoxyalkylene groups, including polyoxyethylene, polyoxypropylene, and the like. Examples of portions that can be protonated to carry one or more positive charges include amino groups, such as polyaminoalkylenes, including ethylene diamines, propylene diamines, butylene diamines and the like, and/or heterocycles, including pyrrolidines, piperidines, piperazines, and other amino groups, each of which can be optionally substituted. Examples of portions that can be deprotonated to carry one or more negative charges include carboxylic acids, such as aspartic acid, glutamic acid, and longer chain carboxylic acid groups, and sulfuric acid esters, such as alkyl esters of sulfuric acid.

Illustrative polyoxyalkylene groups include those of a specific length ranging from about 4 to about 20 polyoxyalkylene (e.g., PEG) groups, such as about 4 to 20, 4 to about 20, or 4 to 20 polyoxyalkylene groups. Illustrative alkyl sulfuric acid esters may also be introduced with click chemistry directly into the backbone. Illustrative L groups comprising polyamines include L groups comprise ethylenediaminetetraacetic acid (EDTA) and diethylenetriamine pentaacetate (DTPA) radicals having the following structure:

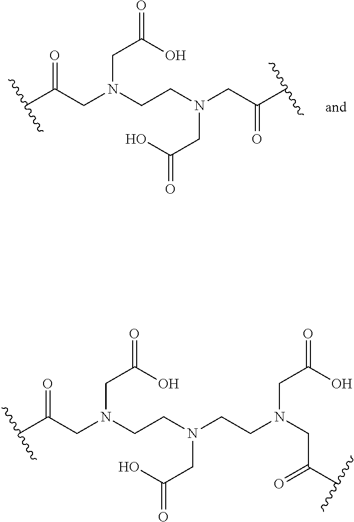 [SMILES: *C(=O)CN(CCN(CC(*)=O)CC(=O)O)CC(=O)O ; *C(=O)CN(CCN(CCN(CC(*)=O)CC(=O)O)CC(=O)O)CC(=O)O]

(poly)peptides:

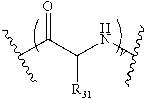

β-amino acids, and the like:

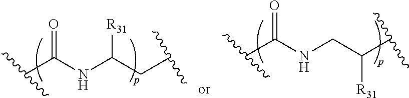

and combinations thereof, wherein each R₃₁ is independently H, alkyl, arylalkyl, heterocyclylalkyl, ureido, aminoalkyl, alkylthio or amidoalkyl, such as in the side chains of naturally occurring amino acids like alanine, valine, leucine, isoleucine, phenylalanine, tyrosine, tryptophan, serine, threonine, asparagine, methionine, lysine, arginine, and histidine. Non-naturally occurring amino acids are also contemplated.

L can comprise a spacer and a cleavable bridge between EVTsmL and AMRL. Alternatively, L can comprise a spacer and a non-cleavable bridge between EVTsmL and AMRL. In certain embodiments, L can make the conjugate more water-soluble. In certain embodiments, L comprises one or more of an amino acid, a PEG monomer, a PEG oligomer, a PEG polymer, or a combination of any of the foregoing. L can comprise an oligomer of peptidoglycans, glycans, anions, or a combination thereof. L can comprise at least one 2,3-diaminopropionic acid, at least one glutamic acid, and/or at least one cysteine.

The terms “non-releasable bridge” or “non-cleavable bridge” are used interchangeably. As used herein, they refer to a linker (L) that cannot be cleaved under extracellular physiological conditions (e.g., a pH-labile, acid-labile, oxidatively-labile, or enzyme-labile bond) (i.e. L is a non-releasable linker). However, such a linker can include bonds that can be cleaved after entry into a cell.

The terms “releasable bridge,” “cleavable bridge,” and “releasable linker” refer to a linker (L) that includes at least one bond that can be cleaved under extracellular physiological conditions (e.g., a pH-labile, acid-labile, oxidatively-labile, or enzyme-labile bond). Releasable groups also include photochemically-cleavable groups. Examples of photochemically-cleavable groups include 2-(2-nitrophenyl)-ethan-2-ol groups and linkers containing o-nitrobenzyl, desyl, trans-o-cinnamoyl, m-nitrophenyl or benzylsulfonyl groups (see, e.g., Dorman and Prestwich, Trends Biotech. 18:64-77 (2000); and Greene and Wuts, Protective Groups in Organic Synthesis, 2nd ed., John Wiley & Sons, New York (1991)).

The cleavable bond or bonds can be present in the interior of a cleavable linker and/or at one or both ends of a cleavable linker. Further, physiological conditions resulting in bond cleavage include standard chemical hydrolysis reactions that occur, for example, at physiological pH, or as a result of compartmentalization into a cellular organelle, such as an endosome having a lower pH than cytosolic pH. Illustratively, in certain embodiments, the bivalent linkers can undergo cleavage under other physiological or metabolic conditions, such as by the action of a glutathione-mediated mechanism. It is appreciated that the lability of the cleavable bond may be adjusted by including functional groups or fragments within the bivalent linker L that are able to assist or facilitate such bond cleavage, also termed anchimeric assistance. The lability of the cleavable bond can also be adjusted by, for example, substitutional changes at or near the cleavable bond, such as including alpha branching adjacent to a cleavable disulfide bond, increasing the hydrophobicity of substituents on silicon in a moiety having a silicon-oxygen bond that can be hydrolyzed, homologating alkoxy groups that form part of a ketal or acetal that can be hydrolyzed, and the like. In addition, it is appreciated that additional functional groups or fragments can be included within the bivalent linker L that are able to assist or facilitate additional fragmentation of the compounds after bond breaking of the releasable linker, when present.

L can include at least one releasable portion. L can include at least two releasable linkers (e.g., cleavable linkers). The choice of a releasable linker or a non-releasable linker can be made independently for each application or configuration of the compounds described herein. The releasable linkers described herein comprise various atoms, chains of atoms, functional groups, and combinations of functional groups. For example, in some embodiments the releasable linker comprises about 1 to about 30 atoms (e.g., about 1 to 30, 1 to about 30, and 1 to 30 atoms), or about 2 to about 20 atoms (e.g., about 2 to 20, 2 to about 20, and 2 to 20 atoms). Lower molecular weight linkers (e.g., those having an approximate molecular weight of about 30 g/mol to about 1,000 g/mol, such as from about 30 g/mol to about 300 g/mol, about 100 g/mol to about 500 g/mol or about 150 g/mol to about 600 g/mol) are also described. Precursors to such linkers can be selected to have either nucleophilic or electrophilic functional groups, or both, optionally in a protected form with a readily cleavable protecting group to facilitate their use in synthesis of the intermediate species.

L can comprise one or more releasable linkers that cleave under the conditions described herein by a chemical mechanism involving beta elimination. Such releasable linkers include beta-thiol, beta-hydroxy, and beta-amino substituted carboxylic acids and derivatives thereof, such as esters, amides, carbonates, carbamates, and ureas. Such linkers also include 2- and 4-thioarylesters, carbamates, and carbonates.

An example of a releasable linker includes a linker of the formula:

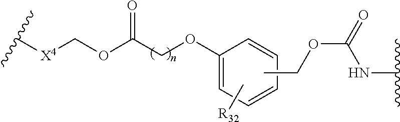

wherein X⁴ is NR, n is an integer selected from 0, 1, 2, and 3, R₃₂ is hydrogen, or a substituent, including a substituent that can stabilize a positive charge inductively or by resonance on the aryl ring, such as alkoxy, and the like. In certain embodiments, the releasable linker can be further substituted.

Assisted cleavage of releasable portions of L can include mechanisms involving benzylium intermediates, benzyne intermediates, lactone cyclization, oxonium intermediates, beta-elimination, and the like. In addition to fragmentation subsequent to cleavage of a releasable portion of L, the initial cleavage of the releasable linker can be facilitated by an anchimerically assisted mechanism. Thus, in the example of a releasable portion of L given above, the hydroxyalkanoic acid, which can cyclize, facilitates cleavage of the methylene bridge by, for example, an oxonium ion, and facilitates bond cleavage or subsequent fragmentation after bond cleavage of the releasable linker. Alternatively, acid catalyzed oxonium ion-assisted cleavage of the methylene bridge can begin a cascade of fragmentation of this illustrative bivalent linker, or fragment thereof. Alternatively, acid-catalyzed hydrolysis of the carbamate may facilitate the beta elimination of the hydroxyalkanoic acid, which can cyclize, and facilitate cleavage of methylene bridge, by for example an oxonium ion. Other chemical mechanisms of bond cleavage under the metabolic, physiological, or cellular conditions described herein can initiate such a cascade of fragmentation as well.

Although the above fragmentation mechanisms are depicted as concerted mechanisms, any number of discrete steps can take place to affect the ultimate fragmentation of a bivalent linker to the final products of the conjugates hereof. For example, the bond cleavage can occur by acid catalyzed elimination of the carbamate moiety, which can be anchimerically assisted by the stabilization provided by either the aryl group of the beta sulfur or disulfide illustrated in the above examples. In those variations of this embodiment, the releasable linker is the carbamate moiety. Alternatively, the fragmentation can be initiated by a nucleophilic attack on the disulfide group, causing cleavage to form a thiolate. The thiolate can intermolecularly displace a carbonic acid or carbamic acid moiety and form the corresponding thiacyclopropane. In the case of the benzyl-containing bivalent linkers, following an illustrative cleavage of the disulfide bond, the resulting phenyl thiolate can further fragment to release a carbonic acid or carbamic acid moiety by forming a resonance-stabilized intermediate. In any of these cases, the releasable nature of the illustrative bivalent linkers described herein can be realized by whatever mechanism is relevant to the chemical, metabolic, physiological, or biological conditions present.

Releasable linkers can comprise a disulfide group. Further examples of releasable linkers comprised in L include divalent radicals comprising alkyleneaziridin-1-yl, alkylenecarbonylaziridin-1-yl, carbonylalkylaziridin-1-yl, alkylenesulfoxylaziridin-1-yl, sulfoxylalkylaziridin-1-yl, sulfonylalkylaziridin-1-yl, or alkylenesulfonylaziridin-1-yl groups, wherein each of the releasable linkers is optionally substituted. Additional examples of releasable linkers comprised in L include divalent radicals comprising methylene, 1-alkoxyalkylene, 1-alkoxycycloalkylene, 1-alkoxyalkylenecarbonyl, 1-alkoxycycloalkylenecarbonyl, carbonylarylcarbonyl, carbonyl(carboxyaryl) carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio or carbonylalkylthio groups, wherein each of the releasable linkers is optionally substituted.

Additional examples of releasable linkers comprised in L include an oxygen atom and methylene, 1-alkoxyalkylene, 1—alkoxycycloalkylene, 1-alkoxyalkylenecarbonyl or 1-alkoxycycloalkylenecarbonyl groups, wherein each of the releasable linkers is optionally substituted. Alternatively, in some embodiments the releasable linker includes an oxygen atom and a methylene group, wherein the methylene group is substituted with an optionally substituted aryl, and the releasable linker is bonded to the oxygen to form an acetal or ketal. Further, in some embodiments the releasable linker includes an oxygen atom and a sulfonylalkyl group, and the releasable linker is bonded to the oxygen to form an alkylsulfonate.

Additional examples of releasable linkers comprised in L include a nitrogen and iminoalkylidenyl, carbonylalkylideniminyl, iminocycloalkylidenyl, and carbonylcycloalkylideniminyl groups, wherein each of the releasable linkers is optionally substituted and the releasable linker is bonded to the nitrogen to form a hydrazone. In some embodiments, the hydrazone is acylated with a carboxylic acid derivative, an orthoformate derivative, or a carbamoyl derivative to form various acylhydrazone releasable linkers.

Additional examples of releasable linkers comprised in L include an oxygen atom and alkylene(dialkylsilyl), alkylene(alkylarylsilyl), alkylene(diarylsilyl), (dialkylsilyl)aryl, (alkylarylsilyl)aryl or (diarylsilyl)aryl groups, wherein each of the releasable linkers is optionally substituted and the releasable linker is bonded to the oxygen to form a silanol.

Additional examples of releasable linkers comprised in L include two independent nitrogens and carbonylarylcarbonyl, carbonyl(carboxyaryl)carbonyl, or carbonyl(biscarboxyaryl)carbonyl. In some embodiments the releasable linker is bonded to the heteroatom nitrogen to form a triazole and bonded to X^{a} or R^{a} via click chemistry (see schemes 1-6 below).

Additional examples of releasable linkers comprised in L include an oxygen atom, a nitrogen, and a carbonylarylcarbonyl, carbonyl(carboxyaryl)carbonyl, or carbonyl(biscarboxyaryl)carbonyl. In some embodiments, the releasable linker forms a triazole, and in some embodiments is bonded to X^{a} or R^{a} via click chemistry.

In some embodiments, L comprises an optionally substituted 1-alkylenesuccinimid-3-yl group and a releasable portion comprising methylene, 1-alkoxyalkylene, 1-alkoxycycloalkylene, 1-alkoxyalkylenecarbonyl or 1-alkoxycycloalkylenecarbonyl groups, each of which can be optionally substituted, to form a succinimid-1-ylalkyl acetal or ketal.

In some embodiments, L comprises carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl or 1-(carbonyltetrahydrofuranyl)succinimid-3-yl, each of which is optionally substituted. In some embodiments, L further comprises an additional nitrogen such that L comprises alkylenecarbonyl, cycloalkylenecarbonyl, carbonylalkylcarbonyl or 1-(carbonylalkyl)succinimid-3-yl groups, each of which is optionally substituted, bonded to the nitrogen to form an amide. In some embodiments, L further comprises a sulfur atom and alkylene or cycloalkylene groups, each of which is optionally substituted with carboxy, and is bonded to the sulfur to form a thiol. In some embodiments, L comprises a sulfur atom and 1-alkylenesuccinimid-3-yl and 1-(carbonylalkyl)succinimid-3-yl groups bonded to the sulfur to form a succinimid-3-ylthiol.

In some embodiments L comprises a nitrogen and a releasable portion comprising alkyleneaziridin-1-yl, carbonylalkylaziridin-1-yl, sulfoxylalkylaziridin-1-yl, or sulfonylalkylaziridin-1-yl, each of which is optionally substituted. In some embodiments, L comprises carbonyl, thionocarbonyl, alkylenecarbonyl, cycloalkylenecarbonyl, carbonylalkylcarbonyl, or 1-(carbonylalkyl)succinimid-3-yl, each of which is optionally substituted, and bonded to the releasable portion to form an aziridine amide.

Examples of L include alkylene-amino-alkylenecarbonyl, alkylene-thio-(carbonylalkylsuccinimid-3-yl), and the like, as further illustrated by the following formulae:

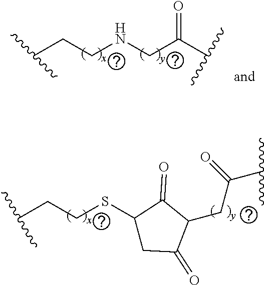

wherein x′ and y′ are each independently 1, 2, 3, 4, or 5.

L can have any suitable assortment of atoms in the chain, including C (e.g., —CH₂—, C(O)), N (e.g., NH, NR^{b}, wherein R^{b} is, e.g., H, alkyl, alkylaryl, and the like), O (e.g., —O—), P (e.g., —O—P(O)(OH)O—), and S (e.g., —S—). For example, the atoms used in forming L can be combined in all chemically relevant ways, such as chains of carbon atoms forming alkyl groups, chains of carbon and oxygen atoms forming polyoxyalkyl groups, chains of carbon and nitrogen atoms forming polyamines, and others, including rings, such as those that form aryl and heterocyclyl groups (e.g., triazoles, oxazoles, and the like). In addition, the bonds connecting atoms in the chain in L may be either saturated or unsaturated, such that for example, alkanes, alkenes, alkynes, cycloalkanes, arylenes, imides, and the like may be divalent radicals that are included in L. Further, the chain-forming L can be substituted or unsubstituted.

Additional examples of L groups include the groups 1-alkylsuccinimid-3-yl, carbonyl, thionocarbonyl, alkyl, cycloalkyl, alkylcycloalkyl, alkylcarbonyl, cycloalkylcarbonyl, carbonylalkylcarbonyl, 1-alkylsuccinimid-3-yl, 1-(carbonylalkyl)succinimid-3-yl, alkylsulfoxyl, sulfonylalkyl, alkylsulfoxylalkyl, alkylsulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranyl)succinimid-3-yl, wherein each group can be substituted or unsubstituted. Any of the aforementioned groups can be L or can be included as a portion of L. In some instances, any of the aforementioned groups can be used in combination (or more than once) (e.g., -alkyl-C(O)-alkyl) and can further comprise an additional nitrogen (e.g., alkyl-C(O)—NH—, —NH-alkyl-C(O)— or —NH-alkyl-), oxygen (e.g., -alkyl-O-alkyl-) or sulfur (e.g., -alkyl-S-alkyl-). Examples of such L groups are alkylcarbonyl, cycloalkylcarbonyl, carbonylalkylcarbonyl, 1-(carbonylalkyl)succinimid-3-yl, and succinimid-3-ylthiol, wherein each group can be substituted or unsubstituted.

In some embodiments, L is formed via click chemistry/click chemistry-derived. The terms “click chemistry” and “click chemistry-derived” generally refer to a class of small molecule reactions commonly used in conjugation, allowing for the joining of substrates of choice with specific molecules. Click chemistry is not a single specific reaction, but instead describes a way of generating products that follow examples in nature, which also generates substances by joining small modular units. In many applications, click reactions join a biomolecule and a reporter molecule. Click chemistry is not limited to biological conditions; the concept of a “click” reaction has been used in pharmacological and various biomimetic applications. However, they have been made notably useful in the detection, localization and qualification of biomolecules.

Click reactions can occur in one pot, are typically not disturbed by water, can generate minimal byproducts, and are “spring-loaded”—characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (for in vivo systems).

Click chemistry examples include examples where L can be derived from copper-catalyzed azide-alkyne cycloaddition (CuAAC), strain-promoted azide-alkyne cycloaddition (SPAAC), inverse electron demand Diels-Alder reaction (IEDDA), and Staudinger ligation (SL). For example, X^{a} and R^{a} can be linked to each other as shown in Schemes 1-6:

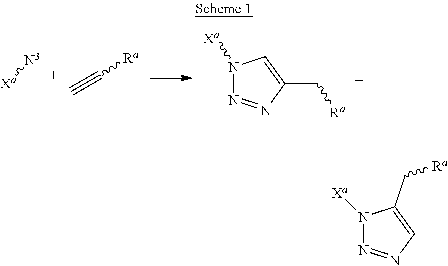

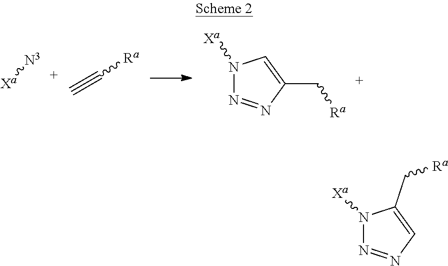

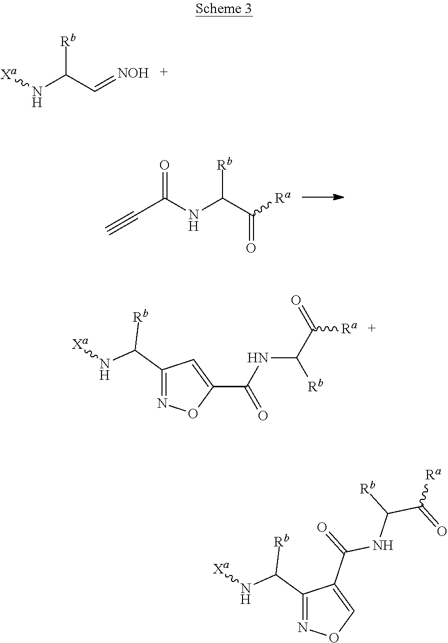

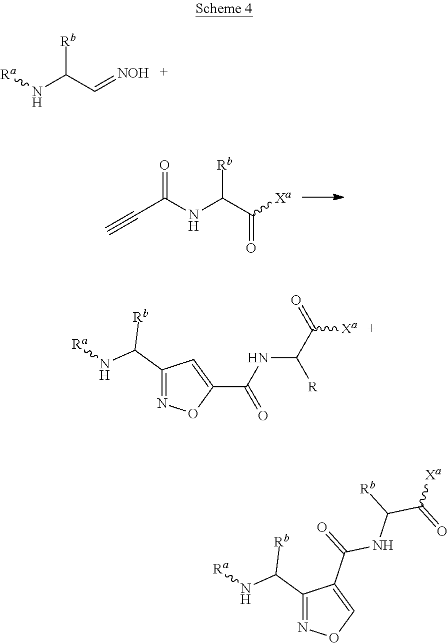

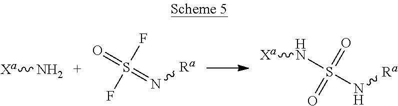

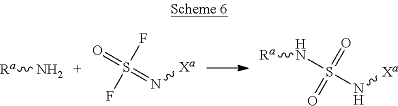

wherein each R^{b} is independently H, alkyl, arylalkyl, -alkyl-S-alkyl or arylalkyl or the side chain of any naturally- or non-naturally occurring amino acid and the like. In Schemes 1-6, the wavy line connected to X^{a} and R^{a} represents a linkage between X^{a} and R^{a} and the groups to which they are attached. It should be appreciated that in Schemes 1-6, the triazole, oxazole, and the —NH—SO₂—NH— groups can be considered to be part of L.

L can comprise at least one linker group, each linker group selected from the group consisting of PEG, alkyl, sugar, and peptide. In some embodiments, the linker is a PEG- (e.g., pegylated-), alkyl-, sugar-, and peptide-based dual linker.

L can comprise a PEG oligomer with 2-16 PEG units. In some embodiments, the linker comprises a PEG oligomer with 12 PEG units.

In certain embodiments, L is:

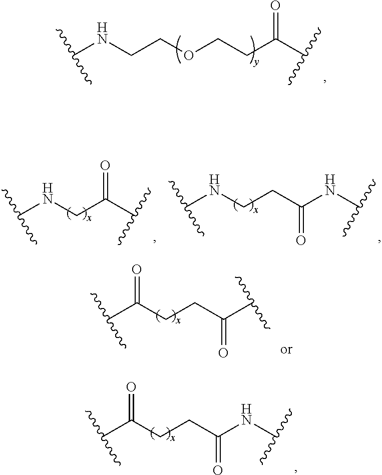

wherein
x″ is an integer from 0 to 10, and
y″ is an integer from 3 to 100. x″ can be an integer from 3 to 10.

In certain embodiments, a “pteroyl” radical, a moiety, or a group has the following structure:

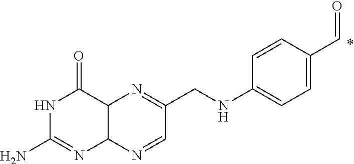

wherein the asterisk (*) denotes the point of attachment of the carbonyl carbon to another chemical group, such as another chemical group in L.

In certain embodiments, L can be:

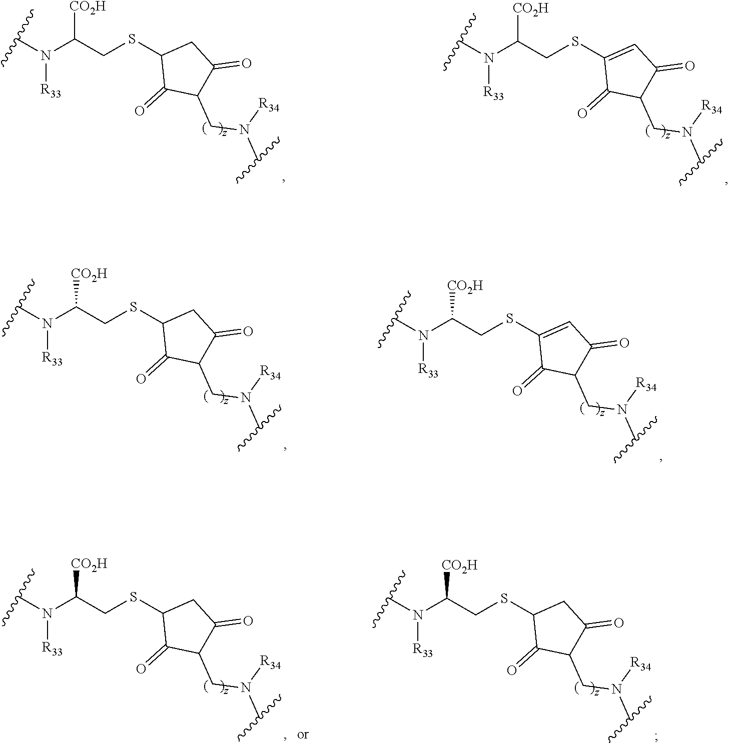

wherein each of R₃₃ and R₃₄ is independently H or C₁-C₆ alkyl; and z is an integer from 1 to 8.
L can be:

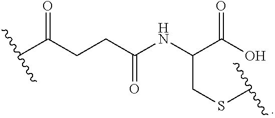 [SMILES: *SCC(NC(=O)CCC(*)=O)C(=O)O]

L can be:

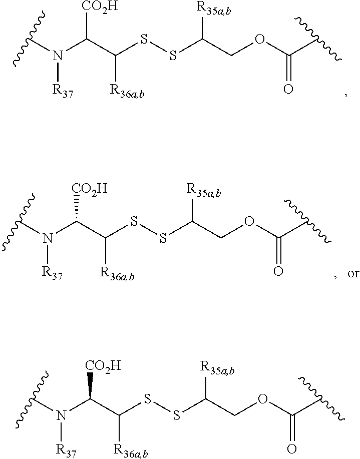

wherein R₃₇ is H or C₁-C₆ alkyl; R₃₅ₐ, R_{35b}, R₃₆ₐ, and R_{36b} each is independently H or C₁-C₆ alkyl.

In certain embodiments, L comprises an amino acid. L can comprise an amino acid selected from the group consisting of Lys, Asn, Thr, Ser, Ile, Met, Pro, His, Gln, Arg, Gly, Asp, Glu, Ala, Val, Phe, Leu, Tyr, Cys, and Trp. In some embodiments, L comprises at least two amino acids independently selected from the group consisting of Glu and Cys. In some embodiments, L comprises Glu-Glu, wherein the glutamic acids are covalently bonded to each other through the carboxylic acid side chains.

In some embodiments, L comprises one or more hydrophilic spacer linkers comprising a plurality of hydroxyl functional groups.

In some embodiments, L comprises at least one 2,3—diaminopropionic acid group, at least one glutamic acid group (e.g., unnatural amino acid D-Glutamic acid), and at least one cysteine group. One example of such a linker is one having the non-natural amino acid, such as a linker having the following formula, or repeating units of the following formula:

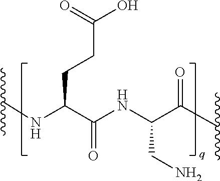

wherein q is an integer from 1 to 10 (e.g., 1 to 3 and 2 to 5).

In some embodiments, L comprises the general formula:

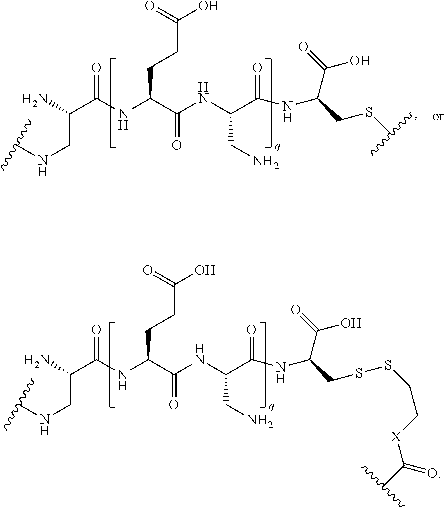

wherein X is O, NH, NR, or S and q is an integer from 1 to 10. In some embodiments, L comprises the formula:

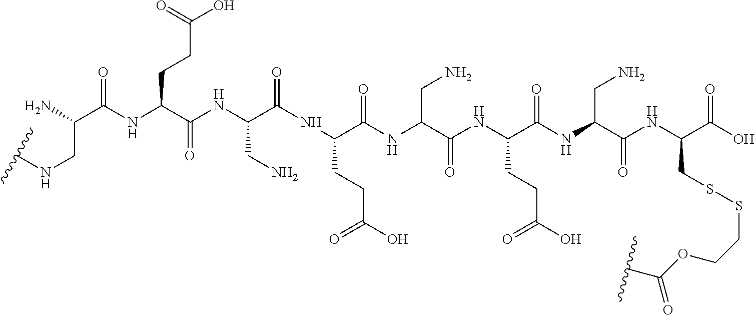 [SMILES: *NC[C@H](N)C(=O)N[C@@H](CCC(=O)O)C(=O)N[C@@H](CN)C(=O)N[C@@H](CCC(=O)O)C(=O)NC(CN)C(=O)N[C@@H](CCC(=O)O)C(=O)N[C@@H](CN)C(=O)N[C@H](CSSCCOC(*)=O)C(=O)O]

wherein the disulfide group is a part of a self-immolative group that can be generically described as a group of the formula —CH₂—S—S—CH₂—.

A release mechanism can involve reduction, oxidation, or hydrolysis. An example of a reduction mechanism includes reduction of a disulfide group into two separate sulfhydryl groups. Thus, for example, a group of the formula —CH₂—S—S—CH₂— would be reduced to two separate groups of the formula —CH₂—SH, such that if the linker were of the formula:

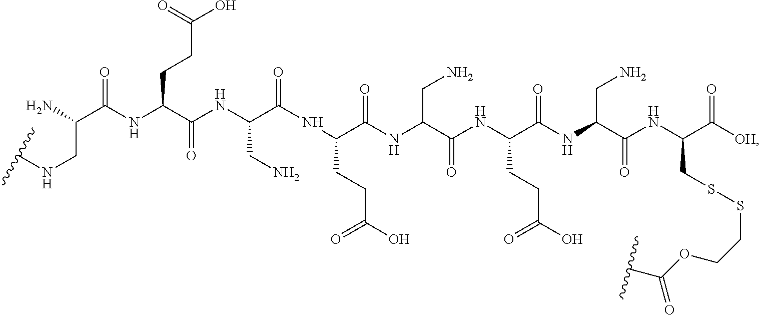 [SMILES: *NC[C@H](N)C(=O)N[C@@H](CCC(=O)O)C(=O)N[C@@H](CN)C(=O)N[C@@H](CCC(=O)O)C(=O)NC(CN)C(=O)N[C@@H](CCC(=O)O)C(=O)N[C@@H](CN)C(=O)N[C@H](CSSCCOC(*)=O)C(=O)O]

the reduction product would be of the formula:

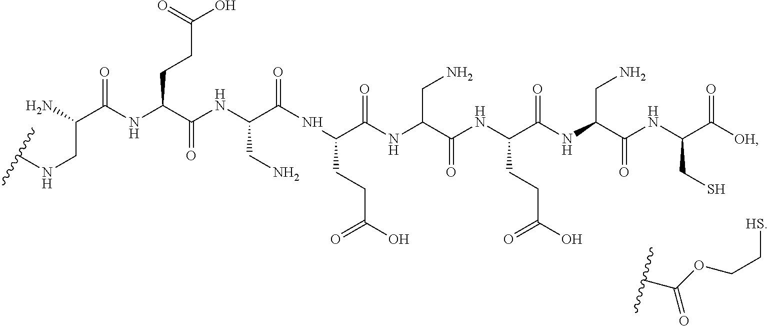 [SMILES: *NC[C@H](N)C(=O)N[C@@H](CCC(=O)O)C(=O)N[C@@H](CN)C(=O)N[C@@H](CCC(=O)O)C(=O)NC(CN)C(=O)N[C@@H](CCC(=O)O)C(=O)N[C@@H](CN)C(=O)N[C@H](CS)C(=O)O ; *C(=O)OCCS]

An example of a self-immolative disulfide linker also includes a sterically protected disulfide bond. The steroid can be attached to the linker via any other suitable self-immolative bond, including via a self-immolative cathepsin cleavable amino acid sequence; via a self-immolative furin cleavable amino acid sequence; via a self-immolative 0-glucuronidase cleavable moiety; via a self-immolative phosphatase cleavable moiety; or via a self-immolative sulfatase cleavable moiety. Multiple self-immolative linkages are also contemplated herein.

In some embodiments, the linker comprises a self-immolative moiety. In some embodiments, the linker comprises a self-immolative disulfide and or sterically protected disulfide bond. In some embodiments, the linker comprises a self-immolative cathepsin-cleavable amino acid sequence. In some embodiments, the linker comprises a self-immolative furin-cleavable amino acid sequence. In some embodiments, the linker comprises a self-immolative (3-glucuronidase-cleavable moiety. In some embodiments, the linker comprises a self-immolative phosphatase-cleavable moiety. In some embodiments, the linker comprises a self-immolative sulfatase-cleavable moiety.

In some embodiments, the linker comprises a phosphate or pyrophosphate group. In some embodiments, the linker comprises a cathepsin B cleavable group. In some embodiments, the cathepsin B cleavable group is valine-citrulline. In some embodiments, the linker comprises a carbamate moiety. In some embodiments, the linker comprises a 0-glucuronide.

In some embodiments, the conjugates described herein include linkers comprising an ester, phosphate, oxime, acetal, pyrophosphate, polyphosphate, disulfide, sulfate, hydrazide, imine, carbonate, carbamate or enzyme-cleavable amino acid sequence.

L can comprise one or more spacer linkers. Spacer linkers can be hydrophilic spacer linkers comprising a plurality of hydroxyl functional groups. A spacer “L” can comprise any stable arrangement of atoms. A spacer comprises one or more L′. Each L′ is independently selected from the group consisting of an amide, ester, urea, carbonate, carbamate, disulfide, amino acid, amine, ether, alkyl, alkene, alkyne, heteroalkyl (e.g., PEG), cycloakyl, aryl, heterocycloalkyl, heteroaryl, carbohydrate, glycan, peptidoglycan, polypeptide, and any combination thereof. In some embodiments, a spacer comprises any one or more of the following units: an amide, ester, urea, carbonate, carbamate, disulfide, amino acid, amine, ether, alkyl, alkene, alkyne, heteroalkyl (e.g., PEG), cycloakyl, aryl, heterocycloalkyl, heteroaryl, carbohydrate, glycan, peptidoglycan, polypeptide, or any combination thereof. In some embodiments, a spacer L or L′ comprises a solubility enhancer or PK/PD modulator W. In some embodiments, a spacer comprises a glycosylated amino acid. In some embodiments, a spacer comprises one or more monosaccharide, disaccharide, polysaccharide, glycan, or peptidoglycan. In some embodiments, a spacer comprises a releasable moiety (e.g., a disulfide bond, an ester, or other moieties that can be cleaved in vivo). In some embodiments, a spacer comprises one or more units such as ethylene (e.g., polyethylene), ethylene glycol (e.g., PEG), ethanolamine, ethylenediamine, and the like (e.g., propylene glycol, propanolamine, propylenediamine). In some embodiments, a spacer comprises an oligoethylene, PEG, alkyl chain, oligopeptide, polypeptide, rigid functionality, peptidoglycan, oligoproline, oligopiperidine, or any combination thereof. In some embodiments, a spacer comprises an oligoethylene glycol or a PEG. A spacer can comprise an oligoethylene glycol. In some embodiments, a spacer comprises a PEG. In some embodiments, a spacer comprises an oligopeptide or polypeptide. In some embodiments, a spacer comprises an oligopeptide. In some embodiments, a spacer comprises a polypeptide. In some embodiments, a spacer comprises a peptidoglycan. In some embodiments, a spacer does not comprise a glycan. In some embodiments, a spacer does not comprise a sugar. In some embodiments, a rigid functionality is an oligoproline or oligopiperidine. In some embodiments, a rigid functionality is an oligoproline. In some embodiments, a rigid functionality is an oligopiperidine. In some embodiments, a rigid functionality is an oligophenyl. In some embodiments, a rigid functionality is an oligoalkyne. In some embodiments, an oligoproline or oligopiperidine has about two up to and including about fifty, about two to about forty, about two to about thirty, about two to about twenty, about two to about fifteen, about two to about ten, or about two to about six repeating units (e.g., prolines or piperidines).

The linker can comprise an albumin ligand. In certain embodiments, the albumin ligand comprises:

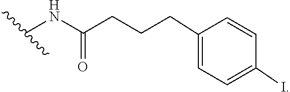 [SMILES: *NC(=O)CCCc1ccc(I)cc1]

The linker can comprise a dimethylcysteine group. The dimethylcysteine group can be linked to a succinimide to form:

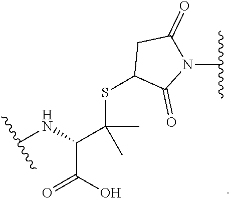 [SMILES: *N[C@@H](C(=O)O)C(C)(C)SC1CC(=O)N(*)C1=O]

A “pteroyl-amino acid” radical, moiety, or group as used herein has the following structure:

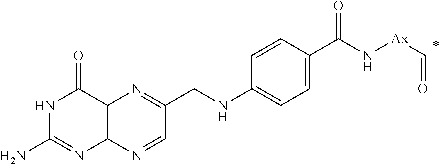

wherein the asterisk denotes the point of attachment of the carbonyl carbon to another chemical group, such as the linker L, and wherein H₂N-Ax-COOH is an amino acid.

As previously described, the linker (L) of the conjugate links the EVTsmL (e.g., a radical of an enveloped virus-targeting, small molecule ligand) and the AMRL (e.g., a radical of an activated macrophage-recruiting ligand).

The AMRL can be a radical of a folate ligand, a folate ligand derivative radical, a folate ligand analog radical, or an antifolate ligand radical. “Folate ligand” means folic acid, dihydrofolate, 5-methyltetrahydrofolate, methylene tetrahydrofolate, and the like, which can bind to a folate receptor. The AMRL can comprise a pteroyl amino acid radical, such as pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine.

In certain embodiments, the AMRL is folic acid, a derivative of folic acid, an analogue of folic acid, or an antifolate. “Antifolate” means a compound that binds to a folate receptor and antagonizes the biological actions of folic acid or one of its naturally occurring forms, such as dihydrofolate, 5-methyltetrahydrofolate, or methylene tetrahydrofolates. Antifolates include, without limitation, inhibitors of dihydrofolate reductase, thymidylate synthase and other enzymes in the folate biosynthesis pathway. Antifolates also include, without limitation, methotrexate, pemetrexed, proguanil, pyrimethamine, ralitrexed, pralatrexate, trimethoprim, and those compounds shown in Table 3.

TABLE 3
Nonclassical Antifolate Analogs

| Li- | |
|---|---|
| gand | Formula |
| aaaa | 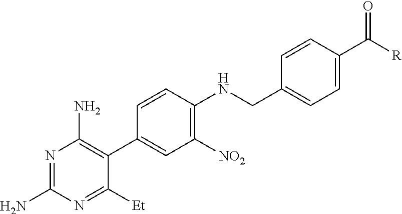 |
| | wherein |
| | R is NH₂, NHMe, NHCH(CO₂Et)(CH₂)₂CO₂Et, |
| | 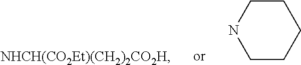 |
| bbbb | 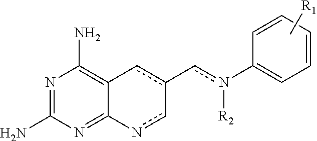 |
| | wherein |
| | R₁ is 3,4,5-(OCH₃)₃, 3,4-(OCH₃)₂, or 4-OCH₃; |
| | R₂ is H, an alkyl chain, or CHO; and |
| | [symbol] is representative of either a single or double bond. |
| cccc | 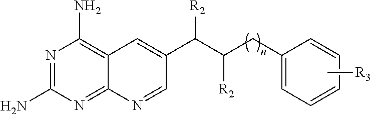 |
| | wherein |
| | n is 0 or 1; |
| | R₂ is each independently an H or an alkyl; |
| | and |
| | R₃ is H, 3′,4′,5′-OMe, 2′,3′,4′-Ome, 2′,4′,5′- |
| | Ome, 2′,4′,6′-Ome, 3′,4′-Ome, 3′,5′-Ome, 2′,5′- |
| | Ome, 2′,3′-C₄H₄, 4′-Ome,2′,3′-C₄H₄, 6′-Ome,2′,3′- |
| | C₄H₄, 4′-O-C₆H₅, or 4′-CONH-L-glutamic acid. |
| dddd | 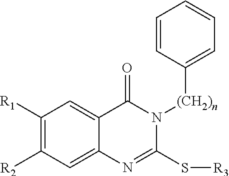 |
| | wherein |
| | n is 0 or 1; |
| | R₁ is CH₃, Cl or OCH₃; |
| | R₂ is H or OCH₃; and |
| | 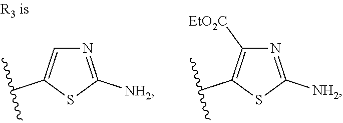 [SMILES: *c1cnc(N)s1 ; *c1sc(N)nc1C(=O)OCC] |
| | 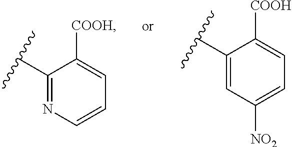 [SMILES: *c1ncccc1C(=O)O ; *c1cc([N+](=O)[O-])ccc1C(=O)O] |
| eeee | 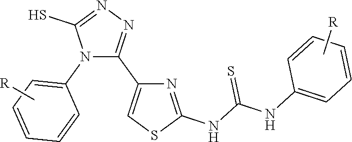 |
| | wherein R is H, 4-Cl, 2-CH₃O, 4-CH₃O, 2,4-(CH₃O)₂, 4-CH₃, or |
| | 4-C₆H₅O. |
| ffff | 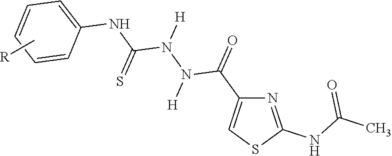 |
| | wherein R is H, 4-Cl, 2-CH₃O, 4-CH₃O, 2,4-(CH₃O)₂, 4-CH₃, or |
| | 4-C₆H₅O. |
| gggg | 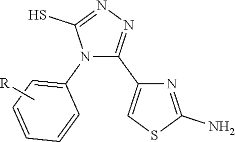 |
| | Wherein R is H, 4-Cl, 2-CH₃O, 4-CH₃O, 2,4-(CH₃O)₂, 4-CH₃, or |
| | 4-C₆H₅O. |
| hhhh | 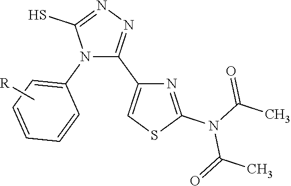 |
| | Wherein R is H, 4-Cl, 2-CH₃O, 4-CH₃O, 2,4-(CH₃O)₂, 4-CH₃, or |
| | 4-C₆H₅O. |
| iiii | 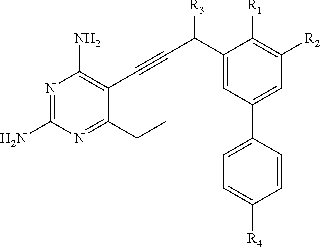 |
| | wherein |
| | R₁ and R₂ are each independently H or OMe; |
| | R₃ is H or an alkyl; and |
| | R₄ is o-COOH or m-COOH or p-COOH. |
| jjjj | 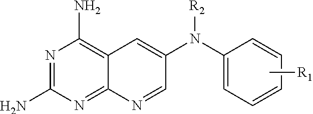 |
| | wherein |
| | R₁ is H, 2′-OMe, 4′-OMe , 2′,5′-diOMe, |
| | 3′,4′,5′-triOMe, 4′-Me, 4′-i-Pr, 3′,4′-(C₄H₄), 2′,3′- |
| | (C₄H₄), 4′-NO₂, 2′,5′-diF or 3′,4′,5′-triF; and |
| | R₂ is H or an alkyl. |

An “analog” or “derivative” with reference to a peptide, polypeptide or protein refers to another peptide, polypeptide or protein that possesses a similar or identical function as the original peptide, polypeptide or protein, but does not necessarily comprise a similar or identical amino acid sequence or structure of the original peptide, polypeptide or protein. An analog preferably satisfies at least one of the following: (a) a proteinaceous agent having an amino acid sequence that is at least 55%, at least 60%, at least 65%, at least 70%, at least 75%, at least 80%, at least 85%, at least 90%, at least 95% or at least 99% identical to the original amino acid sequence; (b) a proteinaceous agent encoded by a nucleotide sequence that hybridizes under stringent conditions to a nucleotide sequence encoding the original amino acid sequence; or (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the original amino acid sequence.

The terms “deaza” and “dideaza” analogs refer to the art-recognized analogs having a carbon atom substituted for one or two nitrogen atoms in the naturally occurring folic acid structure, or analog or derivative thereof. For example, the deaza analogs can include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N10-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate).

The foregoing analogs and/or derivatives are also termed “a folate,” “the folate,” or “folates” reflecting their ability to bind to folate-receptors. Such molecules, when conjugated with exogenous molecules, are effective to enhance transmembrane transport, such as via folate-mediated endocytosis. The foregoing can be used in the folate receptor-binding ligands described herein.

In some embodiments, the conjugate (e.g., compound) can have the structure of formula II:

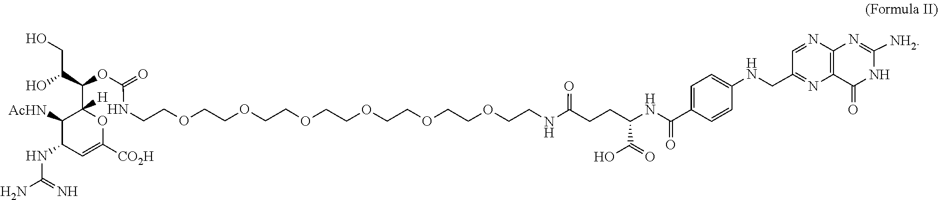 [SMILES: CC(=O)N[C@H]1[C@H]([C@H](OC(=O)NCCOCCOCCOCCOCCOCCOCCNC(=O)CC[C@H](NC(=O)c2ccc(NCc3cnc4nc(N)[nH]c(=O)c4n3)cc2)C(=O)O)[C@H](O)CO)OC(C(=O)O)=C[C@@H]1NC(=N)N]

In some embodiments, the conjugate (e.g., compound) can have the structure of formula III:

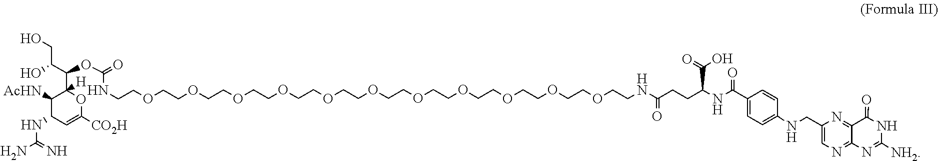 [SMILES: CC(=O)N[C@H]1[C@H]([C@H](OC(=O)NCCOCCOCCOCCOCCOCCOCCOCCOCCOCCOCCOCCNC(=O)CC[C@H](NC(=O)c2ccc(NCc3cnc4nc(N)[nH]c(=O)c4n3)cc2)C(=O)O)[C@H](O)CO)OC(C(=O)O)=C[C@@H]1NC(=N)N]

In some embodiments, the conjugate (e.g., compound) can have the structure of formula IV:

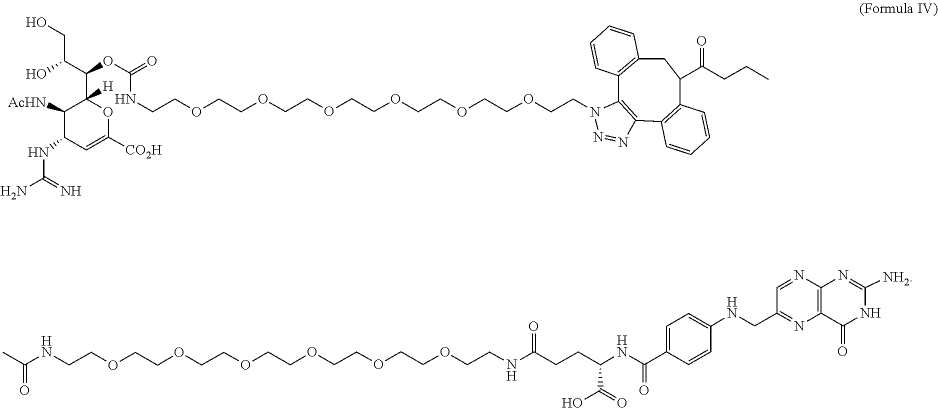

Unless stated otherwise, it is intended that all stereoisomeric forms of the conjugates are contemplated. When the conjugates contain alkene double bonds, and unless specified otherwise, it is intended that this includes both E and Z geometric isomers (e.g., cis or trans). Likewise, all possible isomers, as well as their racemic and optically pure forms, and all tautomeric forms are also intended to be included.

One of ordinary skill in the art will further appreciate that the conjugates can be “deuterated,” meaning one or more hydrogen atoms can be replaced with deuterium.

The conjugates can exist in un-solvated forms as well as solvated forms, including hydrated forms. In general, the solvated forms are equivalent to un-solvated forms. The conjugates can exist in multiple crystalline or amorphous forms. In general, all physical forms are equivalent for the uses contemplated. The formulae include pharmaceutically acceptable salts (e.g., acid addition and base salts), hydrates, and/or solvates.

### Compositions, Routes of Administration, and Dosing

Further provided is a composition comprising an antivirally effective amount of a conjugate or a salt, such as pharmaceutically acceptable salt, thereof. The term “salts” and “pharmaceutically acceptable salts” refer to derivatives of the compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional nontoxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2—acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 21st ed., Lippincott Williams & Wilkins, 2006, e.g., Chapter 38, the disclosure of which is hereby incorporated by reference.

The composition comprising the conjugate can comprise one or more other active agents. Alternatively, the one or more active agents can be included in one or more other compositions, which can be administered by the same route as each other and/or the composition comprising the conjugate or by different routes from each other and/or the composition comprising the conjugate. Examples of other active agents include, for example, other antiviral agents, anti-inflammatory agents, such as nonsteroidal anti-inflammatory agents, and agents that upregulate FRβ expression, such as retinoic acid and curcumin.

The compositions can include one or more suitable production aids or excipients including fillers, binders, disintegrants, lubricants, diluents, flow agents, buffering agents, moistening agents, preservatives, colorants, sweeteners, flavors, and pharmaceutically acceptable carriers. In certain embodiments, provided is a composition comprising a conjugate and an excipient. Excipients are substances added to a pharmaceutical formulation which are not active ingredients. The class of excipients includes diluents (e.g., fillers used to, among other things, increase weight and improve content uniformity in tablets, including starches, hydrolyzed starches, partially pregelatinized starches; other examples of diluents include anhydrous lactose, lactose monohydrate, and sugar alcohols such as sorbitol, xylitol and mannitol).

Excipients generally do not provide any pharmacological activity to the formulation, though they provide chemical and/or biological stability, and release characteristics. Examples of suitable formulations can be found, for example, in Remington, The Science and Practice of Pharmacy, 20th Edition, (Gennaro, A. R., Chief Editor), Philadelphia College of Pharmacy and Science, 2000, which is incorporated by reference in its entirety.

Any suitable excipient (e.g., a pharmaceutically acceptable excipient) can be used in the compositions disclosed herein. Suitable excipients include any and all solvents, dispersion media, coatings, antibacterial and antifungal agents, isotonic and absorption delaying agents that are physiologically compatible.

Supplementary active compounds can also be incorporated into the compositions. Indeed, the conjugates of the compositions can be commingled with other active compounds, and with each other, in a manner such that there is no interaction which would substantially impair the desired pharmaceutical/therapeutic efficiency.

The compositions can be sterile and stable under the conditions of manufacture and storage. The compositions can be formulated as a solution, microemulsion, liposome, or other ordered structure suitable to high drug concentration and, in certain embodiments, can further comprise a carrier. The term “carrier” means one or more compatible solid or liquid fillers, diluents or encapsulating substances which are suitable for administration to a subject (e.g., a human or other vertebrate animal). The carrier can be an organic or inorganic ingredient, natural or synthetic, with which the active ingredient is combined to facilitate the application. For example, the carrier can be a solvent or dispersion medium containing, for example, water, ethanol, polyol (e.g., glycerol, propylene glycol, and liquid PEG), and suitable mixtures thereof. The proper fluidity can be maintained, for example, by the use of a coating, such as lecithin, by the maintenance of the required particle size in the case of dispersion, and by the use of surfactants.

Isotonic agents can be included in the compositions. For example, sugars, polyalcohols such as mannitol, sorbitol, or sodium chloride can be included in the composition. Prolonged absorption of the injectable compositions can be brought about by including in the composition an agent which delays absorption, for example, monostearate salts and gelatin. Moreover, the compounds/conjugates can be formulated in a time-release formulation, for example in a composition that includes a slow-release polymer. The active compounds can be prepared with carriers that will protect the conjugate against rapid release, such as a controlled-release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, and polyglycolic copolymers (PLG). Many methods for the preparation of such formulations are known to those skilled in the art.

For use in therapy or treatment, an effective amount of the conjugate or composition can be administered to a subject by any mode that delivers the conjugate to the desired target. Administering a composition can be accomplished by any means known to the skilled artisan. The compositions can be formulated for administration via one or more of a number of routes including, but not limited to, buccal, cutaneous, direct injection (e.g., into a tumor) epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can be by means of capsule, drops, foams, gel, gum, injection, liquid, patch, pill, porous pouch, powder, tablet, or other suitable means of administration.

In some embodiments, the composition is suitable for parenteral administration. The composition can be formulated as a liquid, e.g., a suspension or a solution. Pharmaceutical formulations (e.g., for parenteral administration) include aqueous solutions of the active conjugates in water-soluble form. Additionally, suspensions of the active conjugates/compounds can be prepared as appropriate oily injection suspensions.

In certain embodiments, a conjugate can be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular, intranasal, and subcutaneous. Suitable means for parenteral administration include needle (including microneedle) injectors, needle-free injectors, and infusion techniques. In embodiments where it is desirable to deliver the conjugates and/or compositions systemically, the conjugate(s) and/or composition can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

In some embodiments, the composition is a sterile aqueous solution or dispersion that can contain carriers or excipients, such as salts, carbohydrates, and buffering agents (preferably at a pH of 3-9). For some applications, the parenteral formulation can be more suitably formulated as a sterile non-aqueous solution or as a dried form (e.g., powder) for the extemporaneous preparation of sterile injectable solutions or dispersion. The compositions can be in powder form for constitution with a suitable vehicle, e.g., sterile pyrogen-free water, before use. Dispersible powders and granules suitable for preparation of an aqueous suspension by the addition of water can provide the active ingredient in admixture with a suspending agent, a dispersing or wetting agent, and one or more preservatives. Additional excipients, for example, coloring agents, also can be present. The use of such media and agents for pharmaceutically active substances is well known in the art. Except insofar as any conventional media or agent is incompatible with the active compound, use thereof in the compositions is contemplated.

A liquid formulation can be adapted for parenteral administration of a conjugate. The preparation of parenteral formulations under sterile conditions, for example, by lyophilization under sterile conditions, can readily be accomplished using standard pharmaceutical techniques well-known to those skilled in the art. The solubility of a conjugate can be increased by the use of appropriate formulation techniques, such as the incorporation of solubility-enhancing agents.

Formulations for parenteral administration can be formulated for immediate and/or modified release. A conjugate can be administered in a time-release formulation, for example in a composition which includes a slow-release polymer. The conjugate can be prepared with a carrier that will protect it against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art.

Sterile injectable solutions can be prepared by incorporating the conjugate(s), alone or in further combination with one or more other active agents, in the required amount in an appropriate solvent with one or a combination of ingredients described above, as required, followed by filtered sterilization. Typically, dispersions are prepared by incorporating the conjugate into a sterile vehicle, which contains a dispersion medium and any additional ingredients of those described above. In the case of sterile powders for the preparation of sterile injectable solutions, the preferred methods of preparation are vacuum-drying and freeze-drying, which yield a powder of the active ingredients plus any additional desired ingredient from a previously sterile-filtered solution thereof, or the ingredients can be sterile-filtered together.

The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid PEG, and the like), and suitable mixtures thereof. The proper fluidity can be maintained, for example, by the use of a coating, such as lecithin, by the maintenance of the required particle size in the case of dispersion, and by the use of surfactants.

Oral forms of administration are also contemplated. The pharmaceutical compositions can be orally administered as a capsule (hard or soft), tablet (film-coated, enteric-coated or uncoated), powder or granules (coated or uncoated) or liquid (solution or suspension). The formulations can be conveniently prepared by any of the methods well-known in the art.

The conjugates can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds, which can be included by admixture, are, for example, medically inert ingredients (e.g., solid and liquid diluent), such as lactose, dextrose saccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or PEGs; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier as described above, for example a natural gum, agar, sodium alginate, pectin, methylcellulose, carboxymethylcellulose, or polyvinyl alcohol.

The amount of active compound (e.g., conjugate) in a composition according to various embodiments can vary according to factors such as the disease state, age, gender, weight, patient history, risk factors, predisposition to disease, administration route, and pre-existing treatment regime (e.g., possible interactions with other medications). Dosage regimens may be adjusted to provide the optimum response. For example, a single bolus may be administered, several divided doses may be administered over time, or the dose may be proportionally reduced or increased as indicated by the exigencies of the situation. Desirably, a therapeutically effective amount is provided, and the amount is sufficient to provide a therapeutic effect to a subject having inflammation, such as inflammation associated with a disease or a disorder, or a prophylactic effect to a subject at risk for developing inflammation associated with a disease or disorder.

Compositions and/or dosage forms for administration can be prepared from a conjugate with a purity of at least approximately 90%, approximately 95%, approximately 96%, approximately 97%, approximately 98%, approximately 99%, or approximately 99.5%. Compositions and/or dosage forms for administration can be prepared from a conjugate with a purity of at least 90%, at least 95%, at least 96%, at least 97%, at least 98%, at least 99%, or at least 99.5%.

“Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms for use with the compositions and methods disclosed herein are dictated by and directly dependent on the unique characteristics of the active compound and the particular therapeutic effect to be achieved, and the limitations inherent in the art of compounding such an active compound for the treatment of sensitivity in individuals. In therapeutic use for treatment of conditions in mammals (e.g., humans) for which the conjugates of the various embodiments described herein, or an appropriate pharmaceutical composition thereof, are effective, the conjugates can be administered in an effective amount.

Any effective regimen for administering the conjugates and/or compositions can be used. The dosages can be single or divided and can be administered according to a wide variety of protocols. “Dose” and “dosage” are used interchangeably herein.

The dosage can be administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the subject requires treatment for a chronic disease or condition, for example, the dosage can be administered for as long as signs and/or symptoms persist. The subject can also require “maintenance treatment” where the patient is receiving dosages every day for months, years, or for life. In addition, the compositions disclosed herein can be to effect prophylaxis of recurring symptoms. For example, the dosage can be administered once or twice a day to prevent the onset of symptoms in subjects at risk, especially for asymptomatic subjects.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred route of administration is intraperitoneal. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect; the composition is applied directly where its action is desired. Administration can be enteral wherein the desired effect is systemic (non-local), composition is given via the digestive tract. Administration can be parenteral, where the desired effect is systemic, composition is given by other routes than the digestive tract. Adjusting the dose to achieve maximal efficacy based on the methods described and other methods as are well-known in the art is well within the capabilities of the ordinarily skilled artisan. Dosage can be adjusted appropriately to achieve desired drug levels, local or systemic, depending upon the mode of administration. For example, the dose for intravenous administration can vary from one order to several orders of magnitude lower per day. If the response in a subject is insufficient at such doses, even higher doses (or effective higher doses by a different, more localized delivery route) can be employed to the extent that subject tolerance permits.

### Methods of Treatment

Still further provided is a method of treating prophylactically or therapeutically a viral infection in a subject. The method comprises administering to the subject an antivirally effective amount of a conjugate of formula I.

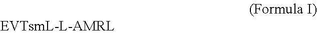

wherein EVTsmL is a radical of an enveloped virus-targeting, small molecule ligand, AMRL is a radical of an activated macrophage-recruiting ligand, and L is a linker that is covalently bound to EVTsmL and AMRL, or a composition comprising an antivirally effective amount of the conjugate.

An “individual,” “subject” or “patient,” refers to a human but can also refer to a non-human animal, such as a mammal.

The term “treating” encompasses therapeutic treatment (e.g., a subject with signs and symptoms of a disease state being treated) and/or prophylactic treatment. Prophylactic treatment encompasses prevention and inhibition or delay of progression of a disease state. If a conjugate, composition or other treatment is administered prior to clinical manifestation of the unwanted condition (e.g., infection or other unwanted state of the subject) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “antivirally effective amount” refers to that amount of one or more conjugates (e.g., a conjugate of the formula (I)), or a composition comprising the same, that inhibits viral infection and/or replication or otherwise elicits the desired biological or medicinal response in the subject that is being sought by a researcher, veterinarian, medical doctor, or other clinician, which includes, but is not limited to, alleviation of the signs and/or symptoms of the disease or disorder being treated (e.g., viral infection). An “antivirally effective amount” with respect to use in treatment refers to an amount of the conjugate in a preparation which, when administered as part of a desired dosage regimen (e.g., to a mammal, such as a human) alleviates a symptom associated with a viral infection, ameliorates a condition of the viral infection, or slows the onset of the viral infection according to clinically acceptable standards or a cosmetic purpose, e.g., at a reasonable benefit/risk ratio applicable to any medical treatment.

A major barrier to curing a viral infection (e.g., an enveloped viral infection) is the latent viral reservoir, which is comprised of transcriptionally limited/silent infected cells that are poorly affected by conventional therapies due, in part, to the limited penetration capabilities of conventional approaches across tissues. Unlike activated CD4⁺ T cells that undergo rapid cell death during initial infection (e.g., due to rapid viral replication kinetics), viral replication kinetics are delayed in non-dividing myeloid cells, which can result in long-lived survival of infected macrophages and macrophage-like cells.

Macrophage activation and dysregulation is a key driver of disease progression across viral infections at least in part because it can result in an inflammatory cytokine cascade that leads to the accumulation of M1 macrophages and activated immune cells and increased systemic inflammation. Further, migration of macrophages into tissues can facilitate the spread of infection throughout the body (e.g., where the macrophages are infected with the virus) and/or further result in proinflammatory cytokines recruiting macrophages and other innate immune cells to the infection site. This cascade compounds macrophage activation and infection, which eventually can confer immune dysregulation and lead to immune exhaustion and/or persistent inflammation in the subject.

The methods hereof leverage the conjugate's ability to target one or both of an enveloped virus and an activated immune cell (e.g., an activated macrophage) to provide an effective and targeted treatment against a viral infection in a subject. When administered, the AMRL of the conjugate can engage an activated immune cell of the subject. In at least one embodiment, when administered to a subject, the AMRL binds FRO, which can be expressed on activated macrophages. In certain embodiments, the AMRL is folic acid, a derivative of folic acid, an analog of folic acid, or an antifolate. In certain embodiments, the AMRL of the conjugate comprises a pteroyl amino acid (e.g., and without limitation, pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine).

With a viral infection, the activated macrophage can itself be infected with the virus and/or be present at, or mobilized to, a site of the viral infection within the subject's body. Accordingly, while the AMRL targets the activated macrophage, the EVTsmL of the conjugate (to which the AMRL is conjugated or tethered) can bind to a cell-surface receptor present on the enveloped virus or a cell infected with an enveloped virus. In this manner, the conjugate hereof forms a bridge between the virus infected cells bound to the EVTsmL and the activated macrophage cells bound to the AMRL. The small molecule of the EVTsmL is advantageous in this context because its small size enables the EVTsmL to penetrate tissues and effectively target a virally infected cell. It also allows for control of the level of immune response provoked as it can, for example, be administered at desired rates and frequencies to elicit a desired level of immune cell engagement.

In certain embodiments, the EVTsmL can bind a viral antigen such as NA or HA on an influenza virus or an influenza virus-infected cell, human gp120 on a HIV or HIV-infected cell, CD38 on a latently infected cell, HBsAg or HBcAg on a HBV or an HBV-infected cell, fusion protein F on a RSV or an RSV-infected cell, spike protein on a coronavirus or a coronavirus-infected cell, or any other viral antigen now known or hereinafter discovered. In certain embodiments, the EVTsmL is zanamivir. In certain embodiments, the EVTsmL is oseltamivir, peramivir, or laninamivir.

In at least one exemplary embodiment, the conjugate administered with the method has the structure of formula II:

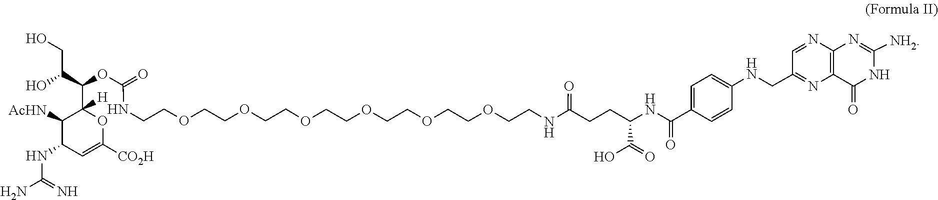 [SMILES: CC(=O)N[C@H]1[C@H]([C@H](OC(=O)NCCOCCOCCOCCOCCOCCOCCNC(=O)CC[C@H](NC(=O)c2ccc(NCc3cnc4nc(N)[nH]c(=O)c4n3)cc2)C(=O)O)[C@H](O)CO)OC(C(=O)O)=C[C@@H]1NC(=N)N]

In at least one exemplary embodiment, the conjugate administered with the method has the structure of formula III:

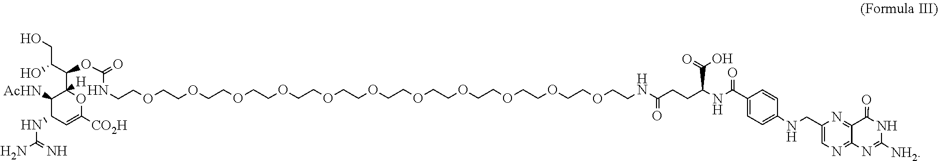 [SMILES: CC(=O)N[C@H]1[C@H]([C@H](OC(=O)NCCOCCOCCOCCOCCOCCOCCOCCOCCOCCOCCOCCNC(=O)CC[C@H](NC(=O)c2ccc(NCc3cnc4nc(N)[nH]c(=O)c4n3)cc2)C(=O)O)[C@H](O)CO)OC(C(=O)O)=C[C@@H]1NC(=N)N]

In at least one exemplary embodiment, the conjugate administered with the method has the structure of formula IV:

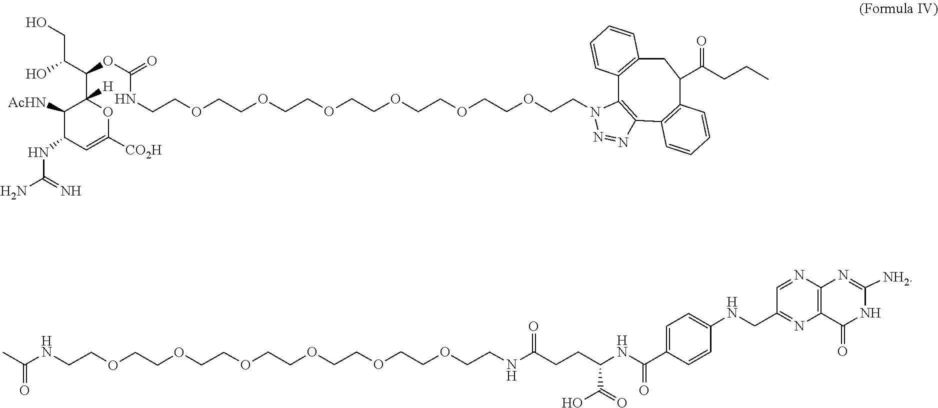

The method can further comprise the administration of one or more other active agents and/or additional therapies. The one or more other active agents can be administered with the conjugate, (e.g., included in the composition comprising the conjugate) or can be administered separately from each other and the conjugate or administered together with each other but separately from the conjugate, e.g., included in one or more other compositions (e.g., when more than one other active agent is to be administered). In certain embodiments, the one or more active agents are antiviral compounds. The one or more other active agents can be administered by the same route as the composition comprising the conjugate or a different route. In this regard, if more than one other active agent is administered, the other active agents can be administered by the same or different routes. It will be appreciated that any other suitable active agents can be administered with the composition as may be suitable and/or desired to treat the subject (e.g., therapeutically or prophylactically).

### Other Definitions

Unless defined otherwise, all technical and scientific terms used herein have the same meaning as commonly understood by one of skill in the chemical and biological arts. Additionally, as used in this specification and the appended claims, the terms “a,” “an,” or “the” are used to include one or more than one unless the context clearly dictates otherwise. The term “or” is used to refer to a nonexclusive “or” unless otherwise indicated. Thus, for example, where a compound/composition is substituted with “an” alkyl or aryl, the compound/composition is optionally substituted with at least one alkyl and/or at least one aryl. In addition, it is to be understood that the phraseology or terminology employed herein, and not otherwise defined, is for the purpose of description only and not of limitation. There also is no intention in the use of such terms and expressions of excluding any equivalents of the features shown and described or portions thereof.

The term “about” as used herein can allow for a degree of variability in a value or range, for example, within 10%, within 5%, or within 1% of a stated value or of a stated limit of a range.

“Alkyl” generally refers to a straight or branched hydrocarbon chain radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₁-C₅ alkyl). An alkyl can comprise one to four carbon atoms (e.g., C₁-C₄ alkyl). An alkyl can comprise one to three carbon atoms (e.g., C₁-C₃ alkyl). An alkyl can comprise one to two carbon atoms (e.g., C₁-C₂ alkyl). An alkyl can comprise one carbon atom (e.g., C₁ alkyl). An alkyl can comprise five to fifteen carbon atoms (e.g., C₅-C₁₅ alkyl). An alkyl can comprise five to eight carbon atoms (e.g., C₅-C₈alkyl). An alkyl can comprise two to five carbon atoms (e.g., C₂-C₅ alkyl). An alkyl can comprise three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkoxy” refers to a radical bonded through an oxygen atom of the formula —O— alkyl, where alkyl is an alkyl chain as defined above.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. Acarbocyclyl can comprise three to ten carbon atoms. A carbocyclyl can comprise five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbomenyl, decalinyl, 7,7-dimethyl-bicyclo[2.2.1]heptanyl, and the like.

The term “comprising” (and related terms such as “comprise” or “comprises” or “having” or “including”) is not intended to exclude an embodiment of any compound, composition, method, process, or the like that may “consist of” or “consist essentially of” the described features. The invention illustratively described herein may be suitably practiced in the absence of any element(s) or limitation(s), which is/are not specifically disclosed herein.

“Halo” or “halogen” refers to bromo, chloro, fluoro or iodo substituents.

The term “heteroalkyl” refers to an alkyl group as defined above in which one or more skeletal carbon atoms of the alkyl are substituted with a heteroatom (with the appropriate number of substituents or valences—for example, —CH₂— can be replaced with —NH— or —O—). For example, each substituted carbon atom is independently substituted with a heteroatom, such as wherein the carbon is substituted with a nitrogen, oxygen, selenium, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). A heteroalkyl is attached to the rest of the molecule at a carbon atom of the heteroalkyl. A heteroalkyl is attached to the rest of the molecule at a heteroatom of the heteroalkyl. A heteroalkyl is a C₁-C₁₈ heteroalkyl. A heteroalkyl is a C₁-C₁₂ heteroalkyl. A heteroalkyl is a C₁-C₆ heteroalkyl. A heteroalkyl is a C₁-C₄ heteroalkyl. Heteroalkyl can include alkoxy, alkoxyalkyl, alkylamino, alkylaminoalkyl, aminoalkyl, heterocycloalkyl, heterocycloalkyl, and heterocycloalkylalkyl, as defined herein.

“Heterocyclyl” refers to a stable 3- to 18-membered non-aromatic ring radical that can comprise two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes aromatic, fused, and/or bridged ring systems. The heteroatoms in the heterocyclyl radical are optionally oxidized. The heterocyclyl radical is partially or fully saturated. Disclosures provided herein of an “heterocyclyl” are intended to include independent recitations of heterocyclyl comprising aromatic and non-aromatic ring structures, unless otherwise stated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, 1,3-benzodioxolyl, 1,4-benzodioxanyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, indolinyl, isoindolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heteroaryl” refers to a radical derived from a 3- to 18-membered aromatic ring radical that can comprise two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quatemized. The heteroaryl is attached to the rest of the molecule through any atom of the ring(s). Examples of heteroaryls include, but are not limited to, azepinyl, acridinyl, benzimidazolyl, benzindolyl, benzofuranyl, benzooxazolyl, benzo[d]thiazolyl, benzothiadiazolyl, benzo[b][1,4]dioxepinyl, benzo[b][1,4]oxazinyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-TH-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e., thienyl).

“Oxo” refers to the ═O radical.

While the concepts of the present disclosure are illustrated and described in detail in the figures and descriptions herein, results in the figures and their description are to be considered as exemplary and not restrictive in character; it being understood that only the illustrative embodiments are shown and described and that all changes and modifications that come within the spirit of the disclosure are desired to be protected. Indeed, the numerous specific details provided are set forth to provide a thorough understanding of the present disclosure. Particular examples may be implemented without some or all of these specific details and it is to be understood that this disclosure is not limited to particular biological systems, particular viruses, particular activated immune cells, or particular organs or tissues, which can, of course, vary, but remain applicable in view of the data provided herein.

The use of headings and subheadings is solely for ease of reference and is not intended to limit the scope of the disclosure under a given heading or subheading to the subject matter set forth there under. Rather, disclosure under any heading or subheading is applicable to all subject matter herein, unless expressly indicated otherwise or contradicted by context.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

Various techniques and mechanisms of the present disclosure will sometimes describe a connection or link between two components. Words such as attached, linked, coupled, connected, tethered and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

### EXAMPLES

The following examples serve to illustrate the present disclosure and are not intended to limit the scope of the claimed invention in any way.

TABLE 4
List of Abbreviations

| | |
|---|---|
| MeOH | Methanol |
| EtOH | Ethanol |
| EtOAc | Ethyl acetate |
| DCM | Dichloromethane |
| ACN | Acetonitrile |
| THF | Tetrahydrofuran |
| TFA | Trifluoroacetic acid |
| TIPS | Triisopropylsilane |
| Py | Pyridine |
| TEA | Triethyl amine |
| DIPEA | N,N-Diisopropylethylamine |
| DCC | N,N′-dicyclohexylcarbodiimide |
| NHS | N-hydroxysuccinimide |
| rt | Room temperature |
| M | Molar |
| DMAP | N,N-Dimethyl aminopyridine |
| PPh₃ | Triphenyl phosphine |
| PEG | Polyethylene glycol |
| ^{t}Bu | tert-Butyl |
| NH₄OAc | Ammonium acetate |
| Equiv. | Equivalence |
| aq. | Aqueous |
| min | Minutes |
| g | Grams |
| mL | Milliliter |
| mmol | Millimoles |
| Qty. | Quantity |
| LC/MS | Liquid chromatography-mass spectrometry |
| ESI | Electron spray inonization |
| m/z | Mass/charge ratio |
| pTSOH | p-Toluene sulfonic acid |
| Na₂CO₃ | Sodium carbonate |
| NaOMe | Sodium methoxide |
| Zan | Zanamivir |
| DNP | Dinitrophenyl |

### Example 1

### Synthesis of Zan-PEG₆-Folate Drug Conjugate (Zan-PEG₆-Fol) (Compound 10)

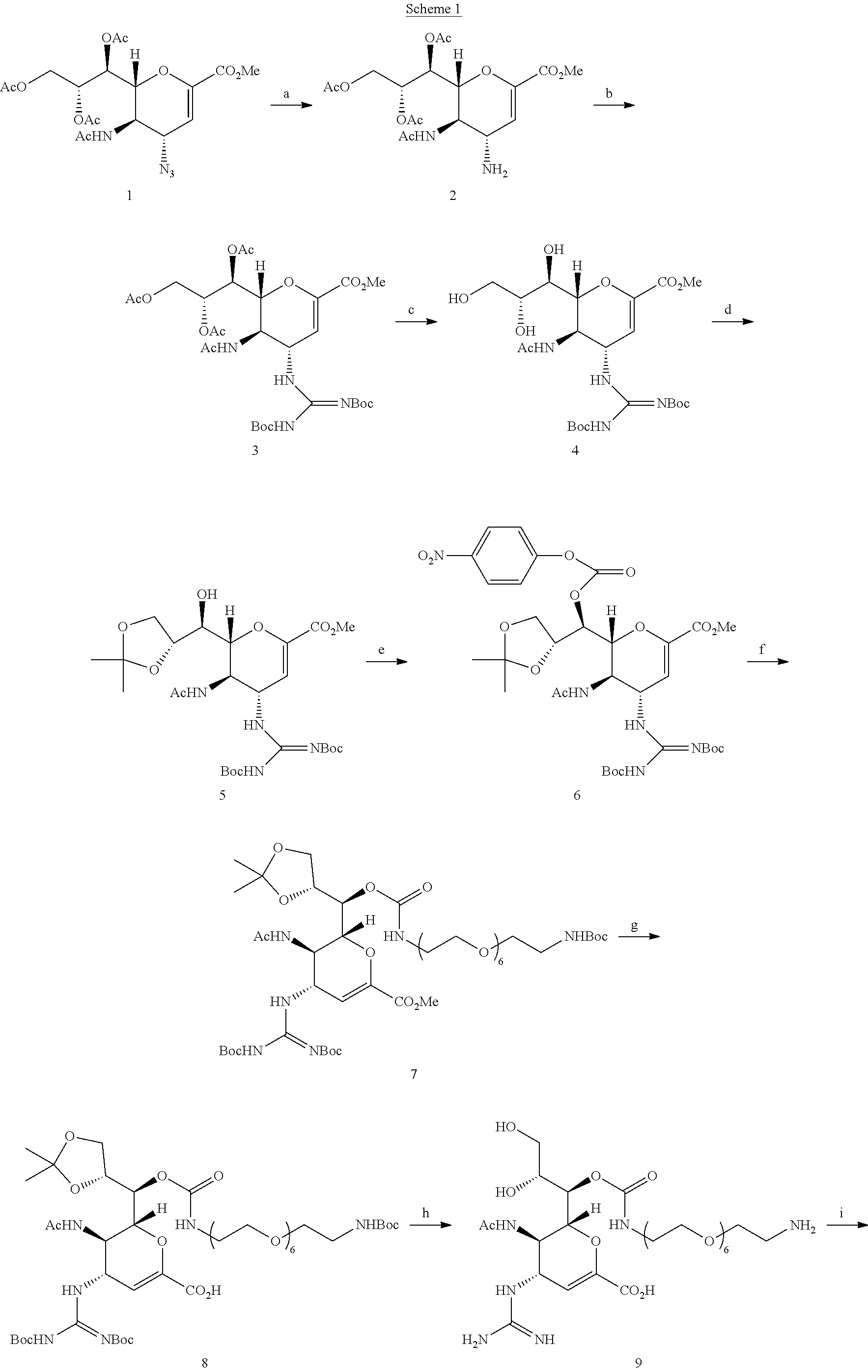

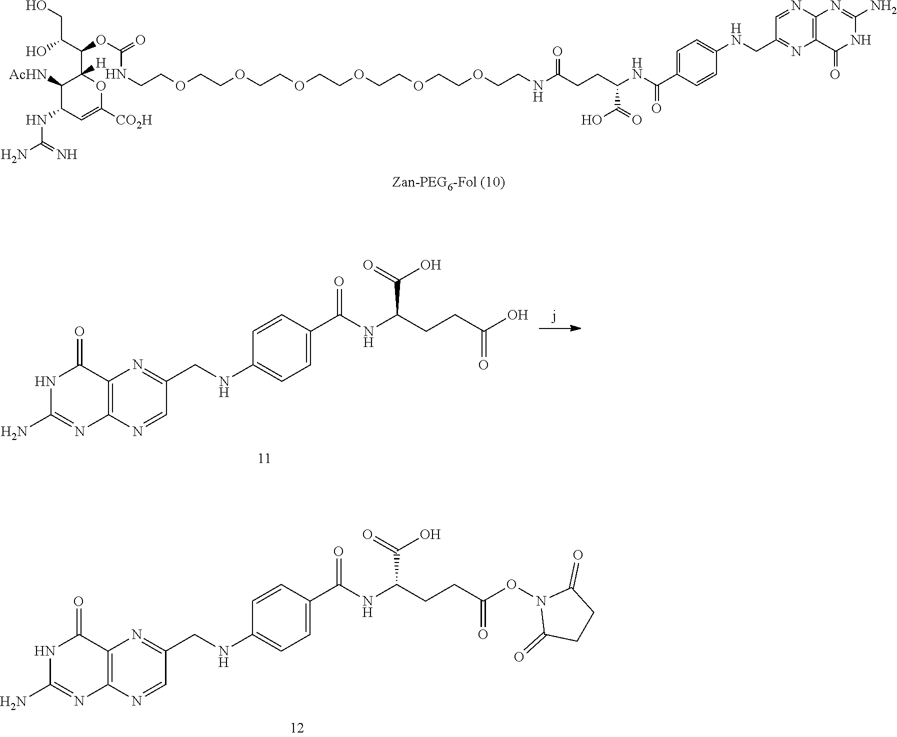 [SMILES: CC(=O)N[C@H]1[C@H]([C@H](OC(=O)NCCOCCOCCOCCOCCOCCOCCNC(=O)CC[C@H](NC(=O)c2ccc(NCc3cnc4nc(N)[nH]c(=O)c4n3)cc2)C(=O)O)[C@H](O)CO)OC(C(=O)O)=C[C@@H]1NC(=N)N ; Nc1nc2ncc(CNc3ccc(C(=O)N[C@H](CCC(=O)O)C(=O)O)cc3)nc2c(=O)[nH]1 ; Nc1nc2ncc(CNc3ccc(C(=O)N[C@@H](CCC(=O)ON4C(=O)CCC4=O)C(=O)O)cc3)nc2c(=O)[nH]1]

The following reagents and conditions were employed in Scheme 1, the steps of which are described in additional detail below: a) PPh₃, THF, H₂O; b) N,N′-Di-Boc-1H-pyrazole-1-carboxamidine, THF, TEA; c) NaOMe, MeOH; d) 2,2-dimethoxypropane, dry Acetone, p-TsOH; e) 4-nitrophenylchloroformate, DMAP, Pyridine, rt; f) Boc-PEG₆-NH₂, DIPEA,THF; g) 1M NaOH (aq), THF; h) TFA, rt; i) Fol-NHS (compound 12), DIPEA, DMSO, rt; j)N-hydroxy succinimide, DCC, DIPEA, DMSO, rt.

Referring to Scheme 1, to synthesize compound 2, a zanamivir derivative (compound 1) was prepared from sialic acid according to previously reported literature methods (see, e.g., Chandler et al. Synthesis of the potent influenza neuraminidase inhibitor 4-guanidino Neu5Ac2en. X-Ray molecular structure of 5-acetamido-4-amino-2, 6-anhydro-3, 4, 5-trideoxy-Derythro-L-gluco-nononic acid. J Chem Society, Perkin Transactions 1: 1173-1180 (1995); Shidmoossavee et al., Chemical insight into the emergence of influenza virus strains that are resistant to Relenza. J Am Chem Soc 135: 13254-13257 (2013); Ying & Gervay-Hague, One-Bead-One-Inhibitor-One-Substrate Screening of Neuraminidase Activity. ChemBioChem 6: 1857-1865 (2005)).

To a solution of zanamivir intermediate (compound 1) (5 g, 11 mmol) in THF (40 mL), triphenyl-phosphine (3.67 g, 14 mmol, 1.27 equiv.) was added and the resulting solution was stirred at rt for 12 hours. Subsequently, water (10 mL) was added and the solution was stirred at rt for another 24 hours. The reaction solution was then concentrated and the crude product was purified by flash column chromatography on a Teledyne CombiFlash Rf+ Lumen (silica gel column, 0-100% EtOAc in hexanes) to give compound 2 as a yellow powder. Compound 2 isolated, 2.89 g; yield, 61.1%.

To synthesize compound 3, triethylamine (1.5 mL) was added toa solution of compound 2 (2.74 g, 6.37 mmol) and N,N′-bis(tertbutoxycarbonyl)-1H-pyrazole-1-carboxamidine (2.57 g, 8.28 mmol, 1.30 equiv.) dissolved in THF (20 mL). The reaction mixture was stirred overnight at rt, and then concentrated and purified by flash column chromatography on a Teledyne CombiFlash Rf+ Lumen (silica gel column, 0-100% EtOAc in hexanes) to give compound 3 as a white solid (4.14 g, 97%).

To synthesize compound 5, NaOMe (2.9 mL, 0.5M, 1.414 mmol) was added to a stirred solution of compound 3 (4.225 g, 6.281 mmol) in anhydrous methanol (70 mL). The reaction mixture was then stirred for 1 hour. Dowex 50XW8 (H+) resin was added to neutralize the reaction mixture and filtered, concentrated to lead compound 4 and was used for the next step without further purification.

2,2-dimethoxypropane (7.7 mL, 6.54 g, 62.81 mmol, 10 equiv.) was added to compound 4 in dry acetone (70 mL), followed by p-toluenesulfonic acid (120 mg, 0.628 mmol, 0.1 equiv.). The resulting mixture was stirred overnight at rt. The reaction mixture was then concentrated and purified by flash column chromatography on a Teledyne CombiFlash Rf+ Lumen (silica gel column, 0-100% EtOAc in hexanes) to give compound 5 as a white solid, Product isolated 2.4 g and yield, 65.2%.

To synthesize compound 6, 4-Dimethylaminopyridine (2.13 g, 17.43 mmol) and 4-nitrophenylchloroformate (3.51 g, 17.43 mmol) was added to a solution of compound 5 (1.46 g, 2.49 mmol) in pyridine (30 mL). The reaction mixture was stirred overnight at rt, and then concentrated and purified by flash column chromatography on a Teledyne CombiFlash Rf+ Lumen (silica gel column, 0-100% EtOAc in hexanes) to give compound 6 as a white solid (1.62 g, 87%).

To synthesize compound 7, NH₂-PEG₆-NHBoc, (0.024 g, 0.48 mmol, 1.2 equiv.) was added to a solution of Activated-Zanamivir, compound 6 (0.075 g, 0.10 mmol) in THF (2.0 mL), followed by DMAP (0.012 g, 0.1 mmol, 1.0 equiv.) at rt under argon gas. The resulting solution was stirred overnight and progress of the reaction was monitored by thin layer chromatography (TLC) and LC/MS. After completion of the reaction, the reaction mixture was concentrated under reduced pressure. The crude product was purified by flash column chromatography on a Teledyne CombiFlash Rf+ Lumen (silica gel column, 5-20% MeOH in DCM) to give compound 7 as a gummy oil (0.085 g, 82%).

Compound 9 was synthesized as follows: Compound 7 (0.08 g, 0.077 mmol) was dissolved in THF (1 mL) and treated drop wise with 1 M NaOH (1 mL). The reaction mixture was stirred at rt for 1 hour at which time LC/MS analysis revealed that deprotection of methyl ester was completed. The reaction mixture was neutralized by adding Dowex® 50WX8 (H+) resin, filtered, and concentrated under reduced pressure. The intermediate crude product (compound 8) was used directly for the next step without further purification.

To the intermediate crude compound (compound 8), TFA (0.5 mL) was added. The reaction mixture was stirred for an hour at rt, with LC/MS confirming the reaction was completed. TFA was removed by rotary evaporation under reduced pressure and the crude product was under vacuum to give compound 9. The total yield over the 2 steps was 97%.

Zan-PEG₆-Fol (compound 10 and also referred to as Formula II in the detailed description) was then synthesized as follows: DIPEA (0.02 μL, 0.117 mmol, 10 equiv.) was added to a solution of compound 9 (0.008 g, 0.012 mmol) and compound 12 (0.008 g, 0.014 mmol, 1.2 equiv.) in DMSO (0.23 mL) at rt under argon gas and stirred for 2-4 hours. Progress of the reaction was monitored by LC/MS. After completion of the reaction, crude mass was purified by reverse phase-HPLC (RP-HPLC) on a C₁₈ column and method gradient as 0-50% B, 20 m/min, 45 minutes (A: 20 mM NH₄OAc, pH 7 buffer and B: acetonitrile) to yield Zan-PEG₆-Fol (compound 10) as a yellow powder (4 mg, 31%). LC/MS [M+H]⁺=1107.12.

To synthesize Fol-NHS ester (compound 12), DCC (0.281 g, 1.359 mmol, 1.2 equiv.) was added to a solution of compound 11 (0.50 g, 1.133 mmol) and N-hydroxysuccinimide (0.143 g, 1.246 mmol, 1.10 equiv.) in DMSO (12 mL) at rt under argon gas and stirred for 12 hours. Progress of the reaction was monitored by LC/MS. After completion of the reaction (as confirmed by LC/MS), the reaction mixture was precipitated in acetone (15×) and the product was isolated by centrifugation. Further, the crude product was washed with acetone (2×50 mL) and EtOAc (1×30 mL), dried under vacuum, and used for the next step without further purification.

### Example 2

### Therapeutic Efficacy of in Protecting Mice from Lethal Influenza Virus Infection

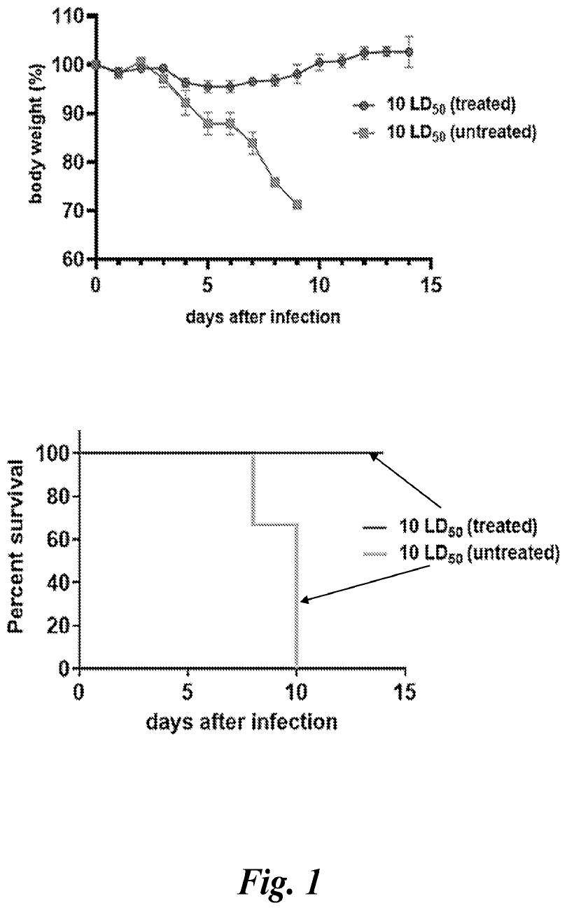

6-8 weeks old female BALB/C mice (n=2/group) were inoculated with 10×LD₅₀ of Influenza A H1N1/PR8/1932 virus. 24 hours after inoculation, two mice were treated intranasally with 1.5 umol/kg of zanamivir-folate (zan-folate) conjugate (compound 10) once daily for five consecutive days. In the control group, two mice were given phosphate buffered saline (PBS) intranasally. The mice in the treatment group did not lose any body weight over time and both the mice survived at the end of two weeks long experiment. On the other hand, in the control group, significant loss of body weight was observed, and both the mice died within 10 days post inoculation. These data (see FIG. 1) show that zan-folate conjugate treated viral infection in vivo.

### Example 3

### Activated Macrophage Migration in a Viral Infection

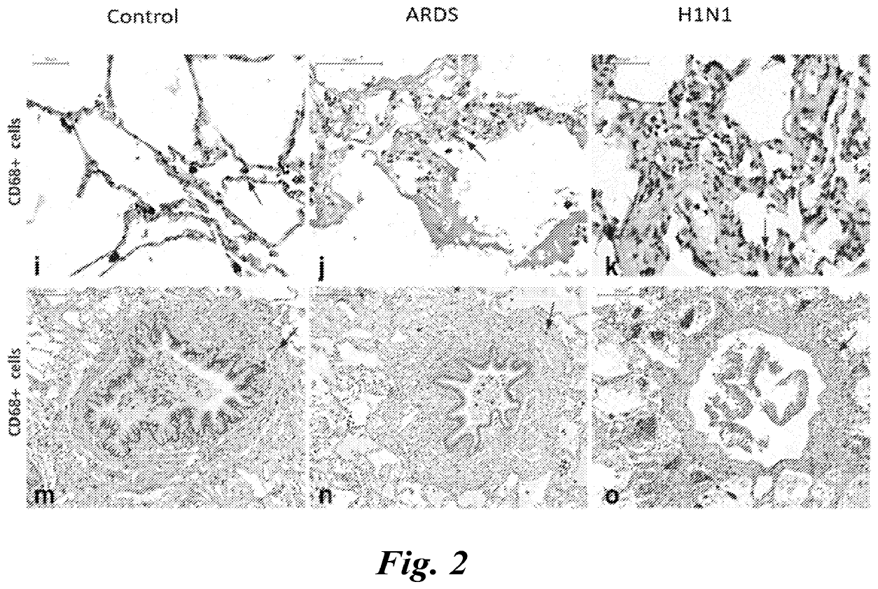

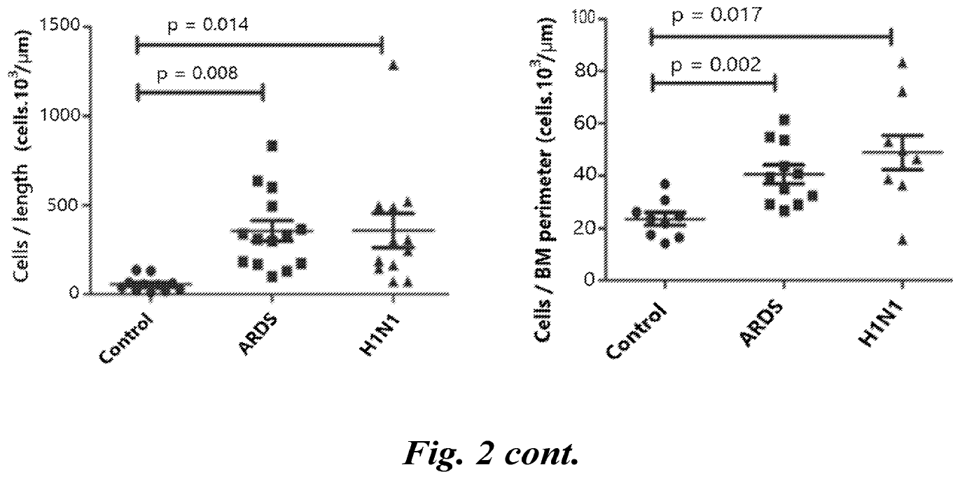

To establish that activated macrophages migrate to a viral infection site, tissue from a control cohort (Control), a cohort of mice experiencing virus-induced acute respiratory distress syndrome (ARDS), and a cohort of mice experiencing an HIN1 infection (H1N1) were assessed. Representative photomicrographs from the Control, ARDS and HIN1 groups show CD68⁺ macrophages in lung parenchyma (i, j, k), and small airways (m, n, o) (see FIG. 2). There was increased expression of CD68⁺ macrophages in the HIN1 and ARDS groups as compared to the Control group, in both the parenchyma and small airways. The arrows indicate positive staining. Graphs show the density of CD68⁺ macrophages in the Control, ARDS and HIN1 groups (one-way ANOVA and Kruskal Wallis test) (Buttignol et al., Respiratory Research 18: 147 (2017); Cancer Immunol Immunother. 58: 1577-1586 (2009); and Arthritis Rheum. 52: 2666-2675 (2005)).

### Example 4

### Dosage Study

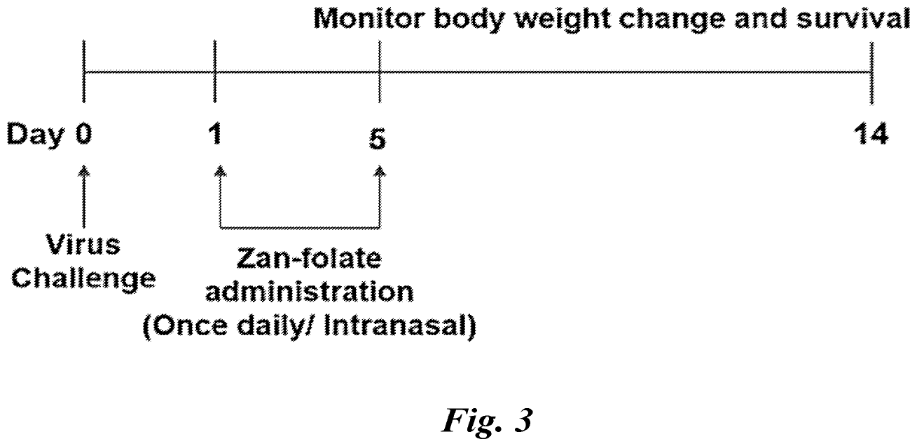

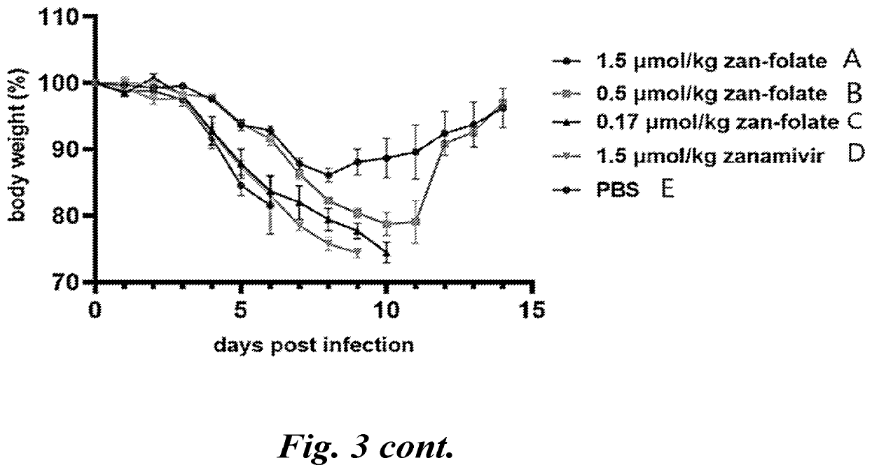

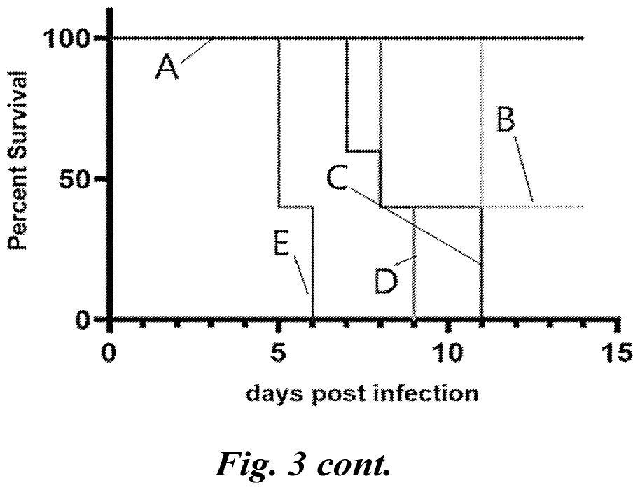

In a dose escalation study, 6-8 weeks old female BALB/C mice (n=5/group) were inoculated with 10×LD₅₀ of Influenza A Puerto Rico8/1934 (H1N1) virus. 24 hours after inoculation, mice in three treatment groups were given 1.5 μmol/kg, 0.5 μmol/kg, and 0.17 μmol/kg of zan-folate conjugate, respectively, once daily for five consecutive days. A dose response pattern was observed as shown in FIG. 3. The survival rate increased with increasing dose of zan-folate and the survival rate was 100% with 1.5 μmol/kg of zan-folate. On the other hand, in the control groups, significant loss of body weight was observed and none of the mice survived at the end of the experiment.

### Example 5

### Linker Variation Study

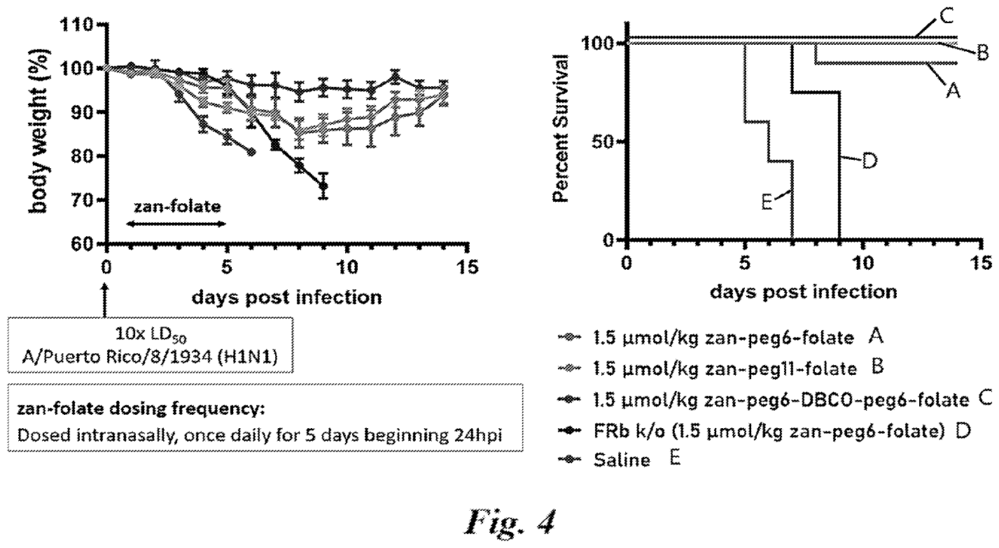

In a linker variation study, 6-8 weeks old female BALB/C mice (n=5/group) were inoculated with 10×LD₅₀ of Influenza A Puerto Rico/8/1934 (H1N1) virus. 24 hours after inoculation, mice were treated with 1.5 μmol/kg zan-PEG₆-folate (compound 10), zan-PEG₁₁-folate (a conjugate of Formula III) or, zan-PEG₆-DBCO-PEG₆-folate (a conjugate of Formula IV) once daily for five consecutive days. As shown in FIG. 4, the PEG₆-DBCO-PEG₆ linker (labelled C in FIG. 4) showed the quickest removal of symptoms and resulted in a 100% survival rate of mice at the end of the experiment. For the zan-conjugates comprising PEG₆ and PEGₙ linkers (labelled A and B, respectively, in FIG. 4), the survival rate was 90% and 100%, respectively, and the weight-loss phenomenon was more pronounced in these linear PEG linkers as compared to a linker containing a rigid dibenzocyclooctyne (DBCO) moiety.

### Example 6

### Conjugate Interaction with Folate Receptors Present on Activated Macrophages

To study if a zan-folate conjugate hereof induces its therapeutic effect through the interaction of folate with folate receptors present on the activated macrophages, folate receptor beta (FRβ) knockout mice were treated with the zan-folate conjugate following influenza infection. None of the mice survived at the end of the experiment, supporting that the conjugates hereof interact with activated macrophages through folate receptors present thereon.

1. A conjugate of formula I: 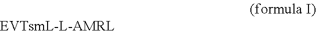
wherein EVTsmL is a radical of an enveloped virus-targeting, small molecule ligand, AMRL is a radical of an activated macrophage-recruiting ligand, and L is a linker that is covalently bound to EVTsmL and AMRL.

2. The conjugate of claim 1, wherein AMRL binds folate receptor R (FRβ) on an activated macrophage.

3. The conjugate of claim 2, wherein EVTsmL binds neuraminidase (NA) or hemagglutinin (HA) on influenza virus or an influenza virus-infected cell.

4. The conjugate of claim 2, wherein EVTsmL is zanamivir.

5. The conjugate of claim 2, wherein EVTsmL is oseltamivir, peramivir, or laninamivir.

6. The conjugate of claim 2, wherein L comprises a spacer and a cleavable bridge between EVTsmL and AMRL.

7. The conjugate of claim 2, wherein L comprises a spacer and a non-cleavable bridge between EVTsmL and AMRL.

8. The conjugate of claim 2, wherein L makes the conjugate more water-soluble.

9. The conjugate of any one of claims 1-8, wherein L comprises one or more of an amino acid, a polyethylene glycol (PEG) monomer, a PEG oligomer, a PEG polymer, or a combination of any of the foregoing.

10. The conjugate of claim 9, wherein L comprises PEG₃, PEG₆, or PEG₁.

11. The conjugate of claim 9 or 10, wherein L further comprises a rigid moiety.

12. The conjugate of claim 9, wherein L further comprises a rigid moiety that is dibenzocyclooctyne (DBCO).

13. The conjugate of claim 12, wherein L comprises PEGₙ-DBCO-PEGₙ, wherein each n independently is an integer between 1-15.

14. The conjugate of claim 13, wherein L is PEG₃-DBCO-PEG₃, PEG₆-DBCO-PEG₆, or PEG₁₁-DBCO-PEG₁₁.

15. The conjugate of any one of claims 1-8, wherein L comprises an oligomer of peptidoglycans, glycans, anions, or a combination thereof.

16. The conjugate of any of claims 2-8, wherein AMRL is folic acid, a derivative of folic acid, an analog of folic acid, or an antifolate ligand radical.

17. The conjugate of claim 1, wherein:
L comprises one or more of an amino acid, a PEG monomer, a PEG oligomer, a PEG polymer, or a combination of any of the foregoing; and
AMRL is folic acid, a derivative of folic acid, an analog of folic acid, or an antifolate.

18. The conjugate of claim 1, wherein:
L comprises an oligomer of peptidoglycans, glycans, anions, or a combination thereof; and
AMRL is folic acid, a derivative of folic acid, an analogue of folic acid, or an antifolate.

19. The conjugate of any of claims 1-8, wherein AMRL comprises a pteroyl amino acid.

20. The conjugate of any one of claims 1-8, wherein AMRL comprises a pteroyl amino acid selected from the group consisting of pteroyl lysine, pteroyl cysteine, pteroyl tyrosine, pteroyl aspartic acid, pteroyl tryptophan, pteroyl serine, pteroyl threonine, pteroyl histidine, pteroyl asparagine, and pteroyl glutamine.

21. The conjugate of claim 1, wherein:
L comprises one or more of an amino acid, a PEG monomer, a PEG oligomer, a PEG polymer, or a combination of any of the foregoing; and
AMRL comprises a pteroyl amino acid.

22. The conjugate of claim 21, wherein the pteroyl amino acid is pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine.

23. The conjugate of claim 1, wherein L comprises an oligomer of peptidoglycans, glycans, anions, or a combination thereof, and AMRL comprises a pteroyl amino acid.

24. The conjugate of claim 23, wherein the pteroyl amino acid is pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine.

25. The conjugate of claim 1, wherein AMRL comprises a pteroyl amino acid.

26. The conjugate of claim 25, wherein the pteroyl amino acid is pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine.

27. The conjugate of claim 1, which has the structure of formula II:

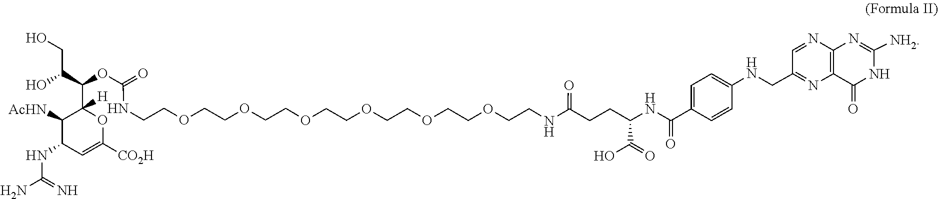 [SMILES: CC(=O)N[C@H]1[C@H]([C@H](OC(=O)NCCOCCOCCOCCOCCOCCOCCNC(=O)CC[C@H](NC(=O)c2ccc(NCc3cnc4nc(N)[nH]c(=O)c4n3)cc2)C(=O)O)[C@H](O)CO)OC(C(=O)O)=C[C@@H]1NC(=N)N]

28. The conjugate of claim 1, which has the structure of formula III:

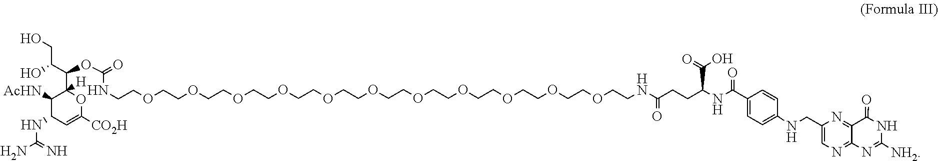 [SMILES: CC(=O)N[C@H]1[C@H]([C@H](OC(=O)NCCOCCOCCOCCOCCOCCOCCOCCOCCOCCOCCOCCNC(=O)CC[C@H](NC(=O)c2ccc(NCc3cnc4nc(N)[nH]c(=O)c4n3)cc2)C(=O)O)[C@H](O)CO)OC(C(=O)O)=C[C@@H]1NC(=N)N]

29. The conjugate of claim 1, which has the structure of formula IV:

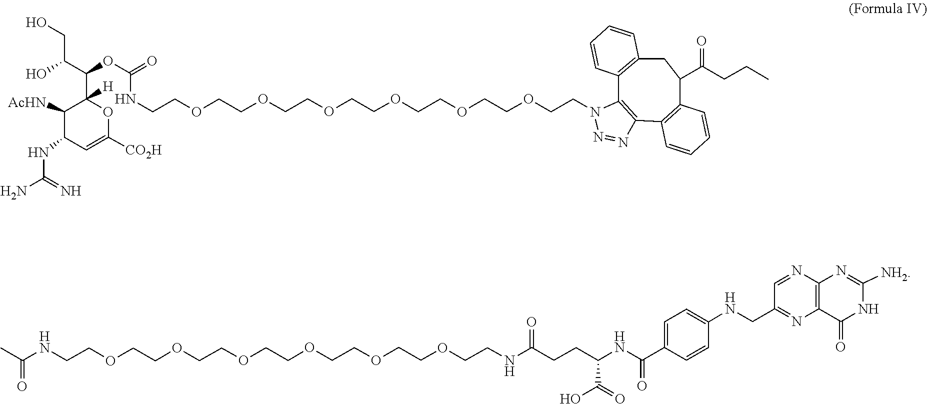

30. A composition comprising an antivirally effective amount of a conjugate of any of claims 1-29.

31. The composition of claim 30, further comprising a pharmaceutically acceptable excipient.

32. The composition of claim 30 or 31, further comprising one or more active agents.

33. A method of treating prophylactically or therapeutically a viral infection in a subject, which method comprises administering to the subject an antivirally effective amount of a conjugate of formula I: 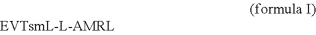
wherein EVTsmL is a radical of an enveloped virus-targeting, small molecule ligand, AMRL is a radical of an activated macrophage-recruiting ligand, and L is a linker that is covalently bound to EVTsmL and AMRL, or a composition comprising an antivirally effective amount of the conjugate.

34. The method of claim 33, wherein AMRL binds FRβ on an activated macrophage.

35. The method of claim 33, wherein EVTsmL binds NA or HA on an influenza virus or an influenza virus-infected cell.

36. The method of claim 33, wherein EVTsmL is zanamivir.

37. The method of claim 33, wherein EVTsmL is oseltamivir, peramivir, or laninamivir.

38. The method of claim 33, wherein L comprises a spacer and a cleavable bridge between EVTsmL and AMRL.

39. The method of claim 33, wherein L comprises a spacer and a non-cleavable bridge between EVTsmL and AMRL.

40. The method of claim 33, wherein L enhances the water-solubility of the conjugate.

41. The method of any one of claims 33-40, wherein L comprises one or more of an amino acid, a PEG monomer, a PEG oligomer, a PEG polymer, or a combination of any of the foregoing.

42. The method of claim 41, wherein L comprises PEG₃, PEG₆, or PEG₁₁.

43. The method of claim 41 or 42, wherein L further comprises a rigid moiety.

44. The method of claim 43, wherein the rigid moiety is DBCO.

45. The method of claim 44, wherein L comprises PEGₙ-DBCO-PEGₙ, wherein each n independently is an integer between 1-15.

46. The method of claim 45, wherein L is PEG₃-DBCO-PEG₃, PEG₆-DBCO-PEG₆, or PEG₁₁-DBCO-PEG₁₁.

47. The method of any one of claims 33-40, wherein L comprises an oligomer of peptidoglycans, glycans, anions, or a combination thereof.

48. The method of any of claims 34-40, wherein AMRL is folic acid, a derivative of folic acid, an analog of folic acid, or an antifolate.

49. The method of claim 33, wherein L comprises one or more of an amino acid, a PEG monomer, a PEG oligomer, a PEG polymer, or a combination of any of the foregoing, and AMRL is folic acid, a derivative of folic acid, an analog of folic acid, or an antifolate.

50. The method of claim 33, wherein:
L comprises an oligomer of peptidoglycans, glycans, anions, or a combination thereof; and
AMRL is folic acid, a derivative of folic acid, an analogue of folic acid, or an antifolate.

51. The method of any of claims 33-40, wherein AMRL comprises a pteroyl amino acid.

52. The method of claim 33-40, wherein AMRL comprises pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine.

53. The method of claim 33, wherein:
L comprises one or more of an amino acid, a PEG monomer, a PEG oligomer, a PEG polymer, or a combination of any of the foregoing; and
AMRL comprises a pteroyl amino acid.

54. The method of claim 53, wherein the pteroyl amino acid is pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine.

55. The method of claim 33, wherein L comprises an oligomer of peptidoglycans, glycans, anions, or a combination thereof, and AMRL comprises a pteroyl amino acid.

56. The method of claim 55, wherein the pteroyl amino acid is pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine.

57. The method of claim 33, wherein AMRL comprises a pteroyl amino acid.

58. The method of claim 57, wherein the pteroyl amino acid is pteroyl lysine, pteroyl cysteine, pteroyl tyrosine, pteroyl aspartic acid, pteroyl tryptophan, pteroyl serine, pteroyl threonine, pteroyl histidine, pteroyl asparagine, or pteroyl glutamine.

59. The method of claim 33, wherein the conjugate has the structure of formula II:

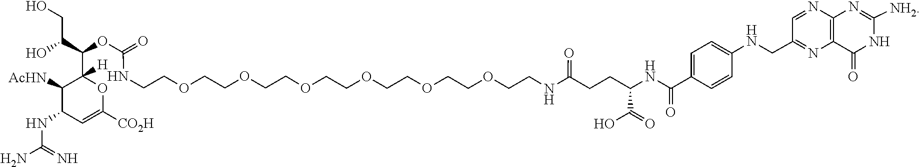 [SMILES: CC(=O)N[C@H]1[C@H]([C@H](OC(=O)NCCOCCOCCOCCOCCOCCOCCNC(=O)CC[C@H](NC(=O)c2ccc(NCc3cnc4nc(N)[nH]c(=O)c4n3)cc2)C(=O)O)[C@H](O)CO)OC(C(=O)O)=C[C@@H]1NC(=N)N]

60. The method of claim 33, wherein the conjugate has the structure of formula III:

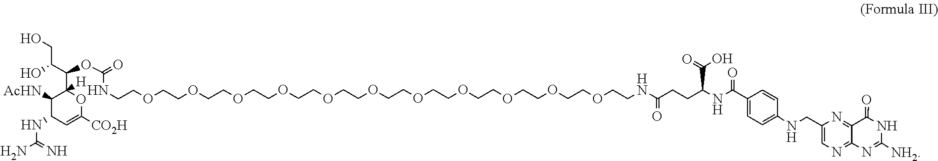 [SMILES: CC(=O)N[C@H]1[C@H]([C@H](OC(=O)NCCOCCOCCOCCOCCOCCOCCOCCOCCOCCOCCOCCNC(=O)CC[C@H](NC(=O)c2ccc(NCc3cnc4nc(N)[nH]c(=O)c4n3)cc2)C(=O)O)[C@H](O)CO)OC(C(=O)O)=C[C@@H]1NC(=N)N]

61. The method of claim 33, which has the structure of formula IV: [chemical structure]
